(19) United States
(12) Patent Application Publication
(10) Pub. No.: US 2022/0050106 A1
(43) Pub. Date: Feb. 17, 2022

(54) METHODS AND KITS FOR DETECTING SARS-CORONAVIRUS-2 ANTIGEN

(71) Applicant: Quanterix Corporation, Billerica, MA (US)

(72) Inventors: Andrew Ball, Billerica, MA (US); Lei Chang, Billerica, MA (US); Syrena Fernandes, Billerica, MA (US); Kevin Hrusovsky, Billerica, MA (US); Joseph Johnson, Billerica, MA (US); Dawn Mattoon, Billerica, MA (US); Tatiana Plavina, Billerica, MA (US); Dandan Shan, Billerica, MA (US); David Wilson, Billerica, MA (US)

(73) Assignee: Quanterix Corporation, Billerica, MA (US)

(21) Appl. No.: 17/400,086

(22) Filed: Aug. 11, 2021

Related U.S. Application Data
(60) Provisional application No. 63/065,974, filed on Aug. 14, 2020.

(51) Int. Cl. G01N 33/569; G01N 33/543

(52) U.S. Cl. CPC G01N 33/56983; G01N 2800/26; G01N 33/54366

(57) ABSTRACT

The present disclosure relates to methods and compositions, e.g., kits, for assessing or detecting SARS-CoV-2 antigen(s), e.g., SARS-CoV-2 nucleocapsid protein (N-protein), in a sample or a blood sample, e.g., serum, plasma, dried blood spots (DBS) and/or a saliva sample. Certain applications and uses of the present methods and compositions, e.g., kits, are also provided.

### RELATED APPLICATION

The present application claims priority to U.S. provisional patent application No. 63/065,974, filed on Aug. 14, 2020, the disclosure of which is incorporated herein by reference in its entirety for all purposes.

### GOVERNMENT SUPPORT

This invention was made with Government support under grant No. HL143541 awarded by the National Institutes of Health. The Government has certain rights in the invention.

### TECHNICAL FIELD

The present disclosure relates to methods and compositions, e.g., kits, for assessing or detecting SARS-CoV-2 antigen(s), e.g., SARS-CoV-2 nucleocapsid protein (N-protein), in a sample or a blood sample, e.g., serum, plasma, dried blood spots (DBS) and/or a saliva sample. Certain applications and uses of the present methods and compositions, e.g., kits, are also provided.

### BACKGROUND

In November 2019, the SARS-CoV-2 (severe acute respiratory syndrome coronavirus-2) emerged in Wuhan, China and since has caused a worldwide pandemic¹. To date, the USFDA has approved or cleared three types of SARS-CoV-2 assays for Emergency Use Authorization: molecular testing or PCR, antibody testing or serology, and antigen testing². Molecular testing for viral RNA is the primary diagnostic modality for active infection, while serology measures anti-SARS-CoV2 antibodies post-infection^{3,4}. Although RT-PCR-based molecular testing for viral RNA in respiratory specimens is the primary diagnostic tool for active infection, concerns have been raised about the risk of false negative results associated with the use of nasopharyngeal swabs⁵. This is especially true in the days before symptom onset; Kucirka et al. have found the probability of a false negative result in an infected person to decrease from 100% on day 1 post-infection 67% on day 4. On day 5, the median time for symptoms to appear, molecular tests still had a 38% probability of producing a false negative result⁶. Furthermore, the complexity, cost, supply chain challenges, and relatively slow turn-around time of RT-PCR results make it unlikely to fulfill large-scale testing required to enable societies to re-open⁷.

Antigen detection has the advantages of a simpler workflow, faster turn-around time, lower cost, and with a supply chain diversified from PCR. However, in general, currently available tests are less sensitive than PCR. A lateral flow assay has been reported to have percent positive agreement (PPA) with qRT-PCR of only 24 to 30%^{8,9}. Two EUA approved antigen tests in the U.S. have claimed sample types of nasopharyngeal or nasal swabs with 96.7% and 84% PPA with PCR and should greatly enhance diagnostic capacity, however they have limitations in several aspects: negative results after day 5 require confirmation with a molecular assay; tests are indicated for use within a seven-day window of infection, limiting the time in which they are useful; they suffer from the same sampling challenges due to reliance on nasal or nasopharyngeal swabs^{10,11}.

SARS-CoV-2 infections can present unusual peripheral symptoms, such as stroke, heart attack, kidney damage, neurological symptoms, and COVID-toe. These clinical manifestations suggest that this respiratory virus can migrate from the lungs into the bloodstream. Mehra et al. first described evidence of SARS-CoV-2 peripheral involvement during post-mortem histological examination of effected tissues, including electron microscopy images of viral inclusion structures in endothelial cells¹². It was hypothesized that SARS-CoV-2 infection may facilitate the induction of endothelitis in multiple organs as a direct consequence of viral involvement. The clinical and histological evidence suggests that SARS-CoV-2 should be, at least transiently, present in blood.

Although venous or capillary blood is a more straightforward matrix to collect than nasopharyngeal swabs, Wölfel et al. have reported that SARS-Cov-2 virus was not detectable in blood using molecular diagnostic techniques¹³. However, recently, Ogata et al measured SARS-CoV-2 antigens and antibodies (S1 antigen, spike antigen, N-protein, and IgG) in venous blood. They hypothesized that detection of viral antigen could be used to stratify patients between mild and severe cases, but that asymptomatic or mild cases would not have measurable levels¹⁴. If true, this would be a distinct difference between SARS-CoV-2 and SARS-CoV, as patients of the latter had measurable levels of N-protein in blood up to 3 weeks after symptom onset, and measurement of N-protein had 94% PPA up to 5 days compared to PCR¹⁵.

Additional and/or improved methods and compositions or kits for assessing or detecting SARS-CoV-2 antigen(s), e.g., SARS-CoV-2 nucleocapsid protein (N-protein), in a sample or a blood sample, e.g., serum, plasma, dried blood spots (DBS) and/or a saliva sample, are needed. The present disclosure provides for methods and compositions or kits to address this and the related needs.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, the present disclosure provides methods and compositions, e.g., kits, for assessing or detecting SARS-CoV-2 antigen(s), e.g., SARS-CoV-2 nucleocapsid protein (N-protein), in a sample or a blood sample, e.g., serum, plasma, dried blood spots (DBS) and/or a saliva sample. In some embodiments, the SARS-CoV-2 antigen(s), e.g., SARS-CoV-2 N-protein, is or are assessed or detected using an immunoassay, e.g., an ultrasensitive immunoassay such as a highly sensitive single molecule array (Simoa) immunoassay. Any suitable Simoa instruments or systems can be used. In some embodiments, Simoa HD-X instruments from Quanterix Corporation, Billerica, Mass., can be used. (See e.g., U.S. Pat. Nos. and patent publication Nos. 8,222,047, 8,236,574, 9,678,068, 8,415,171, 9,952,237, 9,110,025, US-2016-0123969-A1, and US-2018-0003703-A1, which are expressly incorporated by reference in their entireties herein.)

In another aspect, the present disclosure provides a method for quantitatively detecting a severe acute respiratory syndrome coronavirus 2 (SARS-CoV-2) antigen in a sample, which method comprises: a) contacting a sample containing or suspected of containing a SARS-CoV-2 antigen with a capture antibody and a detection antibody, said capture antibody or detection antibody comprising a detectable label, under suitable conditions to allow formation of a sandwich complex comprising said SARS-CoV-2 antigen, if present in said sample, said capture antibody and said detection antibody; and b) assessing a detectable signal from said sandwich complex to assess the amount or level of said SARS-CoV-2 antigen in said sample, wherein said sample comprises a blood sample or a saliva sample, e.g., a blood sample or a saliva sample from a subject, and said method is conducted using a single molecule array immunoassay.

In still another aspect, the present disclosure provides a kit or a system for quantitatively detecting a severe acute respiratory syndrome coronavirus 2 (SARS-CoV-2) antigen in a sample, which kit or a system comprises: a) a capture antibody and a detection antibody, said capture antibody or detection antibody comprising a detectable label, and configured for forming a sandwich complex comprising a SARS-CoV-2 antigen, if present in a sample, said capture antibody and said detection antibody; b) a device or a kit for collecting a blood sample or a saliva sample from a subject; and c) a device, a kit, or a reagent for conducting a single molecule array immunoassay.

Certain applications and uses of the present methods and compositions, e.g., kits, are also provided. For example, the present methods and compositions can be used to differentiate PCR+ from PCR− patients, even if asymptomatic.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an exemplary calibration curve of the Simoa SARS-CoV-2 N Protein Advantage Assay and the Simoa Quantitative SARS-CoV-2 IgG Antibody Test.

FIG. 2 illustrates exemplary Simoa SARS-CoV-2 N Protein measurements in serum/plasma samples from pre-pandemic PCR+ samples. Two sample sets are combined, individual donor samples from Source B (closed symbols) and Longitudinal samples from Source C (open symbols). Samples are binned chronologically: Source B samples are binned as either days from symptom onset or, if asymptomatic, as days from PCR; Source C samples are binned as days from hospitalization. Panel A. Data from the first draw only is shown, excluding subsequent draws in longitudinal sample sets. This data is used to estimate clinical sensitivity and specificity. Panel B. N antigen measurements in all samples, including multiple draws from the same patients, the longitudinal samples from the Source C. This data is used to demonstrate similar sensitivity for week 1 and week 2.

FIG. 3 illustrates exemplary plasma levels of anti-SARS-CoV-2 IgG increases in longitudinal cohort from Source C concurrently with decreases in N-protein (Patients 1-5). Samples from patient 5 were tested with and without DTT treatment, demonstrating a minimal impact of seroconversion on measured N-protein levels. Fitting to the average, normalized concentration yields an estimate for the number of days required for N antigen to decrease to 10% of initial value and IgG to increase to 90% of final value. The estimated times are 15.6 and 7.7 days for N-antigen decrease to 10% and IgG increase to 90% of final plateaus, respectively.

FIG. 4 illustrates exemplary SARS-CoV-2 N-protein measured in capillary blood (dried blood spots (DBS)) from participating SOURCE D residents and staff over two collections, with confirmatory PCR. PCR− samples are denoted in black (●), PCR+ in red ( [symbol] ), and PCR+ asymptomatic or pre-symptomatic in open red (◯). Panel A: N-protein levels over both collections with lines connecting samples from the same donor. Panel B: Donors grouped into PCR−, asymptomatic PCR+, pre-symptomatic PCR+ and symptomatic PCR+, as noted at time of confirmatory PCR. Only the first collection point is represented for each donor.

FIG. 5 illustrates exemplary comparison of N-protein levels from DBS with clinical severity indicators in SOURCE D cohort. (A) SARS-CoV-2 N protein concentrations at the initial sample collections. (B) N protein clearance after one week.

FIG. 6 illustrates exemplary matched serum and plasma samples from the same donors were found to have excellent correlation in N antigen levels between matrices. Twenty matched samples from Source B confirmed to be PCR+ were tested in both serum and K2 EDTA plasma.

FIG. 7 illustrates that an exemplary DTT reduction protocol was established to unmask N-protein bound by antibody in serum by doing a control experiment with recombinant antigen and capture antibody spiked into sample matrix. N-protein concentration measured in serum was reduced after co-spiking with antibody, indicative of epitope masking. Adding DTT to the sample rescued 63% of the signal loss, indicating that this treatment could unmask antigen in seroconverted samples.

FIG. 8 illustrates that exemplary whole blood drawn into a K2 EDTA plasma tube (3 donors) was spiked with known levels of recombinant N-protein. It was then processed into neat plasma and in parallel into Dried Blood Spots using Neoteryx Mitra tips. After extraction, both sample types were measured, showing a correlation of 0.9926. The concentration in DBS was approximately ½ of that in plasma, as expected due to the excluded volume of hematocrit which is separated from plasma.

FIG. 9 illustrates that exemplary whole blood drawn into a K2 EDTA plasma tube (13 donors) was spiked with known levels of Chimeric SARS-COV2-IgG (calibrator for serology assay) or high endogenous samples. It was then processed into neat plasma and in parallel into Dried Blood Spots using Neoteryx Mitra tips. After extraction, both sample types were measured, showing a correlation of 0.9996. The concentration in DBS was approximately ½ of that in plasma, as expected due to the excluded volume of hematocrit which is separated from plasma. This was confirmed by measuring hemoglobin sample levels, converting to hematocrit, and correcting for excluded volume. This changed the fit slope to 0.82.

FIG. 10 illustrates an exemplary Simoa HD-X system or analyzer. (A) Major areas of the instrument. (B) Overhead plan of the chemistry and digitization modules.

FIG. 11 illustrates that exemplary Simoa SARS-CoV-2 N Protein measurements differentiate pre-pandemic from PCR+ donors. a. Shown are pre-pandemic sera and COVID-positive by molecular test (PCR+) plasma binned by day from PCR (BocaBio (triangles), U. Bonn (circles). First timepoint (closed symbol) and subsequent timepoints (open symbol) are shown. Data number is denoted as n=unique donors (total data points). Lines denote median value. b. Concurrent decrease of N-Protein (red) and increase of anti-SARS-CoV-2 spike IgG (blue). Time-series with N-protein peak at day 1 (n=10). c. Time-series with N-protein peak after day 1 (n=3). Data were normalized to max and aligned at peak N-protein. Non-linear regression to the mean is shown in dashed lines. Non-normalized data is shown in FIG. 16.

FIG. 12 illustrates that SARS-CoV-2 N-protein and anti-SARS-CoV-2 IgG in capillary blood (DBS) differentiate COVID PCR− from COVID PCR+ donors. a. NP-Protein in DBS. b. IgG in DBS. Data is binned by day from PCR result. First timepoint (closed symbol) and subsequent timepoints (open symbol) are shown. Data number is denoted as n=unique donors (total data points). Lines denote median value. c. N-Protein over three collections. d. IgG over three collections. Lines connect individual donors over multiple collections for PCR-(blue symbols), PCR+ with symptoms (red symbols), and PCR+ without symptoms (open red symbols) DBS. PCR+n=donors without symptoms (total donors); PCR− n=total donors. Donor 12 is highlighted.

FIG. 13 illustrates that SARS-CoV-2 N-protein and anti-SARS-CoV-2 IgG levels in capillary blood (DBS) correlate with symptom severity indicators. a. SARS-CoV-2 N protein and b. IgG concentrations segregated by symptom severity. First timepoint (closed symbol) and subsequent timepoints (open symbol) are shown. Data number is denoted as n=unique donors (total data points). Lines denote median value. c. Ranking of donors with increasing N-Protein levels, and color-coding associated with disease severity. Data is grouped by donors (n=11), with each bar representing a single sample from 1 of 3 collections (n=11 samples collection 1; n=6 samples collection 2; n=5 samples collection 3). d. IgG for same donors/collections as in panel c.

FIG. 14 illustrates that SARS-CoV-2 N-protein in saliva differentiate COVID PCR− from PCR+ donors and correlate with capillary blood (DBS). a. N-Protein concentration is binned by day from symptom onset. n=individual donors. b. Matched saliva and DBS longitudinal samples from two donors. N-protein in saliva (blue line) and DBS (red line) and IgG in DBS (black line) for the index case (closed symbols) and housemate (lines). Open symbols represent days when symptoms are present for the housemate. c. N-protein in matched saliva (blue symbols) and DBS (red symbols) as a function of cycle threshold (Ct)-values, with exponential fits (solid lines). d. Scatter plot of N-protein in saliva vs. DBS.

FIG. 15 illustrates an exemplary ROC curves for serum/plasma, DBS (within 14 days of positive PCR) and saliva. See Positive/Negative Cutoff in Methods for more information.

FIG. 16 illustrates exemplary SARS-CoV-2 N-protein and anti-spike IgG levels in plasma from the Univ of Bonn cohort. Sixteen individual donors were sampled over multiple timepoints for a total of 141 data points. N-Protein is shown in red, and IgG in blue.

FIG. 17 illustrates that matched serum and plasma samples from the same donors were found to have correlation in N antigen levels between matrices. Twenty matched samples from BocaBio confirmed to be PCR+ were tested in both serum and K2 EDTA plasma.

FIG. 18 illustrates that whole blood drawn into a K2 EDTA plasma tube (3 donors) was spiked with known levels of recombinant N-protein. It was then processed into neat plasma and in parallel into Dried Blood Microsamples (DBS) using Neoteryx Mitra tips. After extraction, both sample types were measured, showing a correlation of 0.99. The concentration in DBS was approximately ½ of that in plasma, as expected due to the excluded volume of hematocrit which is separated from plasma.

FIG. 19. Panel A. A DTT reduction protocol was established to unmask N-protein bound by antibody in serum by doing a control experiment with recombinant antigen and capture antibody spiked into sample matrix. N-protein concentration measured in serum was reduced after co-spiking with antibody, indicative of epitope masking. Adding DTT to the sample rescued 63% of the signal loss, indicating that this treatment could unmask antigen in seroconverted samples. Panel B. Longitudinal plasma samples from Donor 4 Univ of Bonn, treated with and without DTT before testing. Samples were not treated with DTT before testing for IgG. Negligible decrease in N-protein levels were observed with DTT treatment, suggesting that antibody-antigen complexes were not masking N-protein signal, or causing the observed decrease over time. N-Protein is shown in red, N-Protein from samples treated with DTT is shown in orange, and IgG is shown in blue.

FIG. 20 illustrates exemplary N-protein measured in plasma, saliva and nasopharyngeal swabs from commercial sources. The solid lines represent median values (+/−95% confidence interval).

### DETAILED DESCRIPTION

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, including patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, patent applications, published applications or other publications that are herein incorporated by reference, the definition set forth herein prevails over the definition that is incorporated herein by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y.; D. Weir & C. Blackwell, eds., Handbook of Experimental Immunology (1996), Wiley-Blackwell; Cellular and Molecular Immunology (A. Abbas et al., W.B. Saunders Co. 1991, 1994); Current Protocols in Immunology (J. Coligan et al. eds. 1991), all of which are herein incorporated in their entireties by reference for all purposes.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

### A. DEFINITIONS

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

The term “antibody” herein is used in the broadest sense and includes polyclonal and monoclonal antibodies, including intact antibodies and functional (antigen-binding) antibody fragments, including fragment antigen binding (Fab) fragments, F(ab)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are not produced by enzyme digestion of a naturally-occurring intact antibody.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

### B. METHODS FOR QUANTITATIVELY DETECTING A BARS-COV-2 ANTIGEN

In one aspect, the present disclosure provides a method for quantitatively detecting a severe acute respiratory syndrome coronavirus 2 (SARS-CoV-2) antigen, which method comprises: a) contacting a sample containing or suspected of containing a SARS-CoV-2 antigen with a capture antibody and a detection antibody, said capture antibody or detection antibody comprising a detectable label, under suitable conditions to allow formation of a sandwich complex comprising said SARS-CoV-2 antigen, if present in said sample, said capture antibody and said detection antibody; and b) assessing a detectable signal from said sandwich complex to assess the amount or level of said SARS-CoV-2 antigen in said sample, wherein said sample comprises a blood sample or a saliva sample, e.g., a blood sample or a saliva sample from a subject, and said method is conducted using a single molecule array immunoassay.

The present methods can be used to quantitatively detect a SARS-CoV-2 antigen in any suitable blood sample. For example, the present methods can be used to quantitatively detect a SARS-CoV-2 antigen in a plasma, serum, capillary blood, venous blood, dried blood sample or dried blood spot (DBS) sample. In some embodiments, the blood sample is a dried blood spot (DBS) sample.

The sample, e.g., a blood sample or a dried blood spot (DBS) sample, can be collected using any suitable collection device. For example, a dried blood spot (DBS) sample can be collected from a subject using a foamish-tipped collection device or a collection device comprising an absorbent probe, e.g., a foamish-tipped collection device or a collection device that collects a defined amount of blood, e.g., capillary blood, from the subject. In some embodiments, the foamish-tipped collection device or a collection device comprising an absorbent probe can be a Mitra collection device or collection kit from Neoteryx. In some embodiments, the foamish-tipped collection device or a collection device comprising an absorbent probe can be a device for collecting a bodily fluid described and/or claimed in U.S. Pat. No. 10,531,821 B2, US patent publication Nos. US 2017/0071520 A1 or US 2013/0116597 A1 or PCT patent publication No. WO 2013/067520 A1.

In specific embodiments, the blood sampling or collection device used in the present methods can have a holder with a manipulating end and an absorbent probe on the opposing end. (See US patent publication No. US 2017/0071520 A1.) The probe can be of hydrophilic polymer sized to directly absorb a predetermined volume of up to about 30 microliters of blood. Ribs on the holder can position the probe within a compartment of a container to prevent contact with the container. The ribs can also position the probe within extraction wells.

In specific embodiments, the blood sampling or collection device used in the present methods can comprise: an elongated and tapered body extending along a longitudinal axis and having a smaller diameter first end and an opposite, larger diameter second end, the second end forming a conical internal recess which recess extends along a first length of the longitudinal axis; an absorbent probe at the first end of the body; three ribs each extending radially outward from the elongated body beginning adjacent the first end and extending along a second length of the elongated body which second length is greater than half the length of the elongated body, each rib having an outwardly extending position stop facing the first end at the same location along the longitudinal axis. (See US patent publication No. US 2017/0071520 A1.)

The sample, e.g., a blood sample or a dried blood spot (DBS) sample, can also be collected using any suitable collection device from a suitable kit. In some embodiments, the kit described and/or claimed in U.S. Pat. No. 10,531,821 B2 can be used. In specific embodiments, the kit can comprise: a plurality of holders each having an elongated and tapered body extending along a longitudinal axis and having a smaller diameter first end and an opposite, larger diameter second end, the second end forming a conical internal recess which recess extends along a first length of the longitudinal axis that includes at least the second end; an absorbent probe at the first end of the body; a container having a first container portion defining part of two to four separate compartments with each compartment receiving and enclosing a different one of the bodies with each body extending along a compartment longitudinal axis when the entire body and probe are enclosed within the different one of the compartments, each compartment having a wall located to abut a position stop on the holder to position the holder relative to the compartment's longitudinal axis so the absorbent probe does not contact the container; a conical projection in each compartment extending along the compartment's longitudinal axis and sized to mate with the conical internal recess of the holder when the holder is placed in one of the compartments. (See U.S. Pat. No. 10,531,821 B2.)

To be used in the present methods, any suitable amount of a sample, e.g., a blood sample or a saliva sample, can be collected from a subject. For example, about 5 μl, 10 μl, 15 μl, 20 μl, 25 μl, or 30 μl of a sample, e.g., a blood sample or a saliva sample, can be collected from a subject. In some embodiments, about 20 μl of whole blood or capillary blood is collected from the subject.

To be used in the present methods, a sample can be diluted by any suitable fold. For example, a sample can be diluted ranging from about 1 fold to about 1,000 fold, e.g., about 1 fold, 2 folds, 3 folds, 4 folds, 5 folds, 6 folds, 7 folds, 8 folds, 9 folds, 10 folds, 50 folds, 100 folds, 500 folds, 1,000 folds, or any subrange thereof.

Any suitable sample dilution buffer can be used. A sample dilution buffer can comprise any suitable buffering substances. For example, a sample dilution buffer can be a phosphate or Tris buffer. A sample dilution buffer can have any suitable pH, e.g., a pH ranging from about pH 7 to about pH 8, e.g., pH 7.1, pH 7.2, pH 7.3, pH 7.4, pH 7.5, pH 7.6, pH 7.7, pH 7.8, pH 7.9, pH 8, or any subrange thereof. A sample dilution buffer can comprise any suitable salt(s), e.g., sodium chloride (ranging from about 100 mM to about 500 mM, or any subrange thereof), and/or potassium chloride (ranging from about 0 mM to about 5 mM, or any subrange thereof), magnesium chloride (ranging from about 0 mM to about 5 mM, or any subrange thereof). A sample dilution buffer can also comprise a sugar, e.g., sucrose (ranging from about 0% (w/w) to about 2% (w/w), or any subrange thereof) and/or dextrose (ranging from about 0% (w/w) to about 2% (w/w), or any subrange thereof). A sample dilution buffer can also comprise a protein source (ranging from about 0% (w/w) to about 25% (w/w), or any subrange thereof), e.g., bovine serum albumin, human serum albumin, fectal calf serum, goat serum. A sample dilution buffer can also comprise a detergent (ranging from about 0% (w/w) to about 2% (w/w), or any subrange thereof), e.g., Tween-20, Triton X-100, and/or NP-40. A sample dilution buffer can also comprise a denaturant (ranging from about 0 mg/ml to about 5 mg/ml, or any subrange thereof), e.g., urea and/or guanidine hydrochloride. A sample dilution buffer can also comprise a globulin (ranging from about 0 mg/ml to about 5 mg/ml, or any subrange thereof), e.g., mouse IgG, rabbit IgG, goat IgG, rat IgG, bovine gamma, and/or human IgG. A sample dilution buffer can also comprise a heterophilic blocker (ranging from about 0 mg/ml to about 0.5 mg/ml, or any subrange thereof), e.g., HBR, HBR-plus, HAMA blocker, Trublock, and/or Superchemiblock. A sample dilution buffer can comprise one, more or all of the above-described substances. In some embodiments, a sample dilution buffer is a phosphate or Tris buffer having a pH ranging from about pH 7 to about pH 8, and comprises suitable salt(s), sugar(s), protein source(s), detergent(s), denaturant(s), globulin(s), and heterophilic blocker(s), e.g., the salt(s), sugar(s), protein source(s), detergent(s), denaturant(s), globulin(s), and heterophilic blocker(s) described above.

The present methods can also be used to quantitatively detect a SARS-CoV-2 antigen in any suitable saliva sample. In some embodiments, about 5 μl, 10 μl, 15 μl, 20 μl, 25 μl, or 30 μl of saliva sample is collected from the subject.

The present methods can also be used to quantitatively detect any suitable SARS-CoV-2 antigen. For example, the present methods can be used to quantitatively detect a SARS-CoV-2 polypeptide, or a fragment thereof. In some embodiments, the present methods can be used to quantitatively detect one or more SARS-CoV-2 polypeptide selected from S (spike) polypeptide, E (envelope) polypeptide, M (membrane) polypeptide, N (nucleocapsid) polypeptide, or a fragment thereof. In specific embodiments, the present methods can be used to quantitatively detect the SARS-CoV-2 N (nucleocapsid) polypeptide, or a fragment thereof.

Any suitable capture antibody and/or the detection antibody can be used in the present methods. For example, the capture antibody and/or the detection antibody can be an antibody, or a fragment thereof, that specifically binds to the SARS-CoV-2 antigen. In some embodiments, the capture antibody and/or the detection antibody is an antibody, or a fragment thereof, that specifically binds to one or more SARS-CoV-2 polypeptide selected from S (spike) polypeptide, E (envelope) polypeptide, M (membrane) polypeptide, N (nucleocapsid) polypeptide, or a fragment thereof. In specific embodiments, the capture antibody and/or the detection antibody is an antibody, or a fragment thereof, that specifically binds to the N (nucleocapsid) polypeptide, or a fragment thereof.

Any suitable form(s) of antibody can be used in the present methods. For example, the capture antibody and/or the detection antibody can be a polyclonal antibody, a monoclonal antibody, or a fragment thereof. In specific embodiments, in a method for quantitatively detecting a SARS-CoV-2 N (nucleocapsid) polypeptide, or a fragment thereof, the capture antibody is a monoclonal antibody, e.g., a mouse monoclonal antibody Catalog number: 40143-MM05 from Sino Biological, and the detection antibody is a monoclonal antibody, e.g., a rabbit monoclonal antibody Catalog number: 40143-R004 from Sino Biological, or vice versa.

The antibodies can be used at any suitable level or concentration in the present method for quantitatively detecting a SARS-CoV-2 antigen, e.g., SARS-CoV-2 N (nucleocapsid) polypeptide. In some embodiments, the capture antibody can be used at level or concentration ranging from about 0.15 mg/ml to about 2 mg/ml, e.g., at about 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, or any subrange thereof. In some embodiments, the detection antibody can be used at level or concentration ranging from about 0.1 mg/ml to about 3 mg/ml, e.g., at about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3.0 mg/ml, or any subrange thereof.

Any suitable detectable label can be used in the present methods. For example, a colorimetric, a radioactive, an enzymatic, a luminescent or a fluorescent label can be used in the present methods.

The present methods can be used to quantitatively detect a SARS-CoV-2 antigen in sample from any suitable subject. For example, the subject can be a mammal. In some embodiments, the mammal is a human. In some embodiments, the mammal is non-human mammal, e.g., a non-human primate such as a monkey, a rabbit, or a rodent.

The present methods can use any suitable form of single molecule array immunoassay. In some embodiments, conducting the single molecule array immunoassay in the present methods comprises: forming the sandwich complex comprising the SARS-CoV-2 antigen, the capture antibody and the detection antibody on microparticles; applying the microparticles to a microfluidic device comprising an array of femtoliter reaction wells and petitioning a single microparticle comprising the sandwich complex in a femtoliter reaction well; and assessing a detectable signal or signals from the petitioned microparticles to assess the amount or level of the SARS-CoV-2 antigen in the sample. (See e.g., U.S. Pat. Nos. and patent publication Nos. 8,222,047, 8,236,574, 9,678,068, 8,415,171, 9,952,237, 9,110,025, US-2016-0123969-A1, and US-2018-0003703-A1, which are expressly incorporated by reference in their entireties herein.)

The present methods can further comprise contacting a sample with a reducing agent, e.g., DTT, to disassociate a SARS-CoV-2 antigen, e.g., SARS-CoV-2 N (nucleocapsid) polypeptide, from an antibody in a sample.

In some embodiments, the present methods can be used to quantitatively detect SARS-CoV-2 N (nucleocapsid) polypeptide, or a fragment thereof, in a blood sample, e.g., a plasma, serum, dried blood sample or dried blood spot (DBS) sample. In specific embodiments, the present methods can be used to quantitatively detect SARS-CoV-2 N (nucleocapsid) polypeptide, or a fragment thereof, in a blood sample, and to achieve a specificity ranging from about 80% to about 100%, e.g., a specificity of about 80%, 85%, 90%, 91%, 92%, 93%, 94%, 95%, 96%, 97%, 98%, 99%, 100%, or any subrange thereof. In specific embodiments, the present methods can be used to quantitatively detect SARS-CoV-2 N (nucleocapsid) polypeptide, or a fragment thereof, in a blood sample, and to achieve a sensitivity ranging from about 80% to about 100%, e.g., a specificity of about 80%, 85%, 90%, 91%, 92%, 93%, 94%, 95%, 96%, 97%, 98%, 99%, 100%, or any subrange thereof. In specific embodiments, the present methods can be used to quantitatively detect SARS-CoV-2 N (nucleocapsid) polypeptide, or a fragment thereof, in a blood sample, and to achieve a precision (or CV) ranging from about 0% to about 30%, e.g., a precision (or CV) of about 0%, 5%, 10%, 15%, 20%, 25%, 30%, or any subrange thereof. In specific embodiments, the present methods can be used to quantitatively detect SARS-CoV-2 N (nucleocapsid) polypeptide, or a fragment thereof, in a blood sample, and to achieve a precision (or CV) ranging from about 0% to about 10%, or from about 5% to about 10%. In specific embodiments, the present methods can be used to quantitatively detect SARS-CoV-2 N (nucleocapsid) polypeptide, or a fragment thereof, in a blood sample, and to achieve a detection cut-off from about 0.2 pg/ml to about 10 pg/ml, e.g., a detection cut-off of about 0.2 pg/ml, 0.5 pg/ml, 1 pg/ml, 2 pg/ml, 3 pg/ml, 4 pg/ml, 5 pg/ml, 6 pg/ml, 7 pg/ml, 8 pg/ml, 9 pg/ml, 10 pg/ml, or a fragment thereof.

In some embodiments, the present methods can be used to quantitatively detect SARS-CoV-2 N (nucleocapsid) polypeptide, or a fragment thereof, in a saliva sample. In specific embodiments, the present methods can be used to quantitatively detect SARS-CoV-2 N (nucleocapsid) polypeptide, or a fragment thereof, in a saliva sample, and to achieve a specificity ranging from about 80% to about 100%, e.g., a specificity of about 80%, 85%, 90%, 91%, 92%, 93%, 94%, 95%, 96%, 97%, 98%, 99%, 100%, or any subrange thereof. In specific embodiments, the present methods can be used to quantitatively detect SARS-CoV-2 N (nucleocapsid) polypeptide, or a fragment thereof, in a saliva sample, and to achieve a sensitivity ranging from about 80% to about 100%, e.g., a specificity of about 80%, 85%, 90%, 91%, 92%, 93%, 94%, 95%, 96%, 97%, 98%, 99%, 100%, or any subrange thereof. In specific embodiments, the present methods can be used to quantitatively detect SARS-CoV-2 N (nucleocapsid) polypeptide, or a fragment thereof, in a saliva sample, and to achieve a precision (or CV) ranging from about 0% to about 30%, e.g., a precision (or CV) of about 0%, 5%, 10%, 15%, 20%, 25%, 30%, or any subrange thereof. In specific embodiments, the present methods can be used to quantitatively detect SARS-CoV-2 N (nucleocapsid) polypeptide, or a fragment thereof, in a blood sample, and to achieve a precision (or CV) ranging from about 0% to about 10%, or from about 5% to about 10%. In specific embodiments, the present methods can be used to quantitatively detect SARS-CoV-2 N (nucleocapsid) polypeptide, or a fragment thereof, in a saliva sample, and to achieve a detection cut-off from about 1 pg/ml to about 5 pg/ml, e.g., e.g., a detection cut-off of about 1 pg/ml, 2 pg/ml, 3 pg/ml, 4 pg/ml, 5 pg/ml, or a fragment thereof.

The present methods can further comprise quantitatively detecting an antibody of the subject to SARS-CoV-2. The antibody of the subject to SARS-CoV-2 can be quantitatively detected using any suitable methods, kits or systems. In some embodiments, The antibody of the subject to SARS-CoV-2 can be quantitatively detected using any methods kits or systems described and/or claimed in U.S. patent application Ser. No. 17/243,502, which is expressly incorporated by reference in its entirety herein.

The present methods can further comprise quantitatively detecting any suitable type of antibody of the subject to SARS-CoV-2. For example, the present methods can further comprise quantitatively detecting a class of IgG antibody of the subject to SARS-CoV-2, e.g., a class of IgG antibody of the subject to SARS-CoV-2 N (nucleocapsid) polypeptide, or a fragment thereof. (See e.g., U.S. patent application Ser. No. 17/243,502.)

The present methods can be used for any suitable purposes or applications. For example, the present methods can be used to aid or facilitate diagnosis, prognosis, risk assessment, stratification and/or treatment monitoring of SARS-CoV-2 infection in a subject, and/or for research and drug/vaccine discovery, and/or development for treating or preventing SARS-CoV-2 infection. In some embodiments, the present methods can be used to assess SARS-CoV-2 infection and/or recovery status in a subject, e.g., among the following: 1) no infection; 2) infection, asymptomatic; 3) infection, pre-symptomatic; 4) infection, symptomatic; or 5) infection, recovered.

### C. KITS AND SYSTEMS FOR QUANTITATIVELY DETECTING A SARS-COV-2 ANTIGEN

In another aspect, the present disclosure provides a kit or a system for quantitatively detecting a severe acute respiratory syndrome coronavirus 2 (SARS-CoV-2) antigen in a sample, which kit or a system comprises: a) a capture antibody and a detection antibody, said capture antibody or detection antibody comprising a detectable label, and configured for forming a sandwich complex comprising a SARS-CoV-2 antigen, if present in a sample, said capture antibody and said detection antibody; b) a device or a kit for collecting a blood sample or a saliva sample from a subject; and c) a device, a kit, or a reagent for conducting a single molecule array immunoassay.

The present kits or systems can be used to quantitatively detect a SARS-Cod′-2 antigen in any suitable blood sample. For example, the present kits or systems can be used to quantitatively detect a SARS-Codi-2 antigen in a plasma, serum, capillary blood, venous blood, dried blood sample or dried blood spot (DBS) sample. In some embodiments, the blood sample is a dried blood spot (DBS) sample.

The present kits or systems can comprise any suitable device or a kit for collecting a blood sample or a saliva sample from a subject. For example, the present kits or systems can comprise any suitable device or a kit for collecting a dried blood spot (DBS) sample. In some embodiments, the present kits or systems can comprise a foamish-tipped collection device or a collection device comprising an absorbent probe, e.g., a foamish-tipped collection device or a collection device that collects a defined amount of blood, e.g., capillary blood, from the subject. In some embodiments, the foamish-tipped collection device or a collection device comprising an absorbent probe can be a Mitra collection device or collection kit from Neoteryx. In some embodiments, the foamish-tipped collection device or a collection device comprising an absorbent probe can be a device for collecting a bodily fluid described and/or claimed in U.S. Pat. No. 10,531,821 B2, US patent publication Nos. US 2017/0071520 A1 or US 2013/0116597 A1 or PCT patent publication No. WO 2013/067520 A1.

In specific embodiments, the blood sampling or collection device used in the present kits or systems can have a holder with a manipulating end and an absorbent probe on the opposing end. (See US patent publication No. US 2017/0071520 A1.) The probe can be of hydrophilic polymer sized to directly absorb a predetermined volume of up to about 30 microliters of blood. Ribs on the holder can position the probe within a compartment of a container to prevent contact with the container. The ribs can also position the probe within extraction wells.

In specific embodiments, the blood sampling or collection device used in the present kits or systems can comprise: an elongated and tapered body extending along a longitudinal axis and having a smaller diameter first end and an opposite, larger diameter second end, the second end forming a conical internal recess which recess extends along a first length of the longitudinal axis; an absorbent probe at the first end of the body; three ribs each extending radially outward from the elongated body beginning adjacent the first end and extending along a second length of the elongated body which second length is greater than half the length of the elongated body, each rib having an outwardly extending position stop facing the first end at the same location along the longitudinal axis. (See US patent publication No. US 2017/0071520 A1.)

In some embodiments, the present kits or systems can comprise the kit described and/or claimed in U.S. Pat. No. 10,531,821 B2 can be used. In specific embodiments, the kit can comprise: a plurality of holders each having an elongated and tapered body extending along a longitudinal axis and having a smaller diameter first end and an opposite, larger diameter second end, the second end forming a conical internal recess which recess extends along a first length of the longitudinal axis that includes at least the second end; an absorbent probe at the first end of the body; a container having a first container portion defining part of two to four separate compartments with each compartment receiving and enclosing a different one of the bodies with each body extending along a compartment longitudinal axis when the entire body and probe are enclosed within the different one of the compartments, each compartment having a wall located to abut a position stop on the holder to position the holder relative to the compartment's longitudinal axis so the absorbent probe does not contact the container; a conical projection in each compartment extending along the compartment's longitudinal axis and sized to mate with the conical internal recess of the holder when the holder is placed in one of the compartments. (See U.S. Pat. No. 10,531,821 B2.)

To be used with the present kits or systems, any suitable amount of a sample, e.g., a blood sample or a saliva sample, can be collected from a subject. For example, about 5 μl, 10 μl, 15 μl, 20 μl, 25 μl, or 30 μl of a sample, e.g., a blood sample or a saliva sample, can be collected from a subject. In some embodiments, about 20 μl of whole blood or capillary blood is collected from the subject.

The present kits or systems can also be used to quantitatively detect a SARS-CoV-2 antigen in any suitable saliva sample. In some embodiments, about 5 μl, 10 μl, 15 μl, 20 μl, 25 μl, or 30 μl of saliva sample is collected from the subject.

The present kits or systems can comprise any suitable capture antibody and/or the detection antibody. For example, the capture antibody and/or the detection antibody can be an antibody, or a fragment thereof, that specifically binds to the SARS-CoV-2 antigen. In some embodiments, the capture antibody and/or the detection antibody is an antibody, or a fragment thereof, that specifically binds to one or more SARS-CoV-2 polypeptide selected from S (spike) polypeptide, E (envelope) polypeptide, M (membrane) polypeptide, N (nucleocapsid) polypeptide, or a fragment thereof. In specific embodiments, the capture antibody and/or the detection antibody is an antibody, or a fragment thereof, that specifically binds to the N (nucleocapsid) polypeptide, or a fragment thereof.

The present kits or systems can comprise any suitable form(s) of antibody. For example, the capture antibody and/or the detection antibody can be a polyclonal antibody, a monoclonal antibody, or a fragment thereof. The detection antibody can comprise any suitable detectable label, e.g., a colorimetric, a radioactive, an enzymatic, a luminescent or a fluorescent label. In specific embodiments, in a method for quantitatively detecting a SARS-CoV-2 N (nucleocapsid) polypeptide, or a fragment thereof, the capture antibody is a monoclonal antibody, e.g., a mouse monoclonal antibody Catalog number: 40143-MM05 from Sino Biological, and the detection antibody is a monoclonal antibody, e.g., a rabbit monoclonal antibody Catalog number: 40143-R004 from Sino Biological, or vice versa.

In some embodiments, the present kits or systems can further comprise a sample dilution buffer. A sample dilution buffer can comprise any suitable buffering substances. For example, a sample dilution buffer can be a phosphate or Tris buffer. A sample dilution buffer can have any suitable pH, e.g., a pH ranging from about pH 7 to about pH 8, e.g., pH 7.1, pH 7.2, pH 7.3, pH 7.4, pH 7.5, pH 7.6, pH 7.7, pH 7.8, pH 7.9, pH 8, or any subrange thereof. A sample dilution buffer can comprise any suitable salt(s), e.g., sodium chloride (ranging from about 100 mM to about 500 mM, or any subrange thereof), and/or potassium chloride (ranging from about 0 mM to about 5 mM, or any subrange thereof), magnesium chloride (ranging from about 0 mM to about 5 mM, or any subrange thereof). A sample dilution buffer can also comprise a sugar, e.g., sucrose (ranging from about 0% (w/w) to about 2% (w/w), or any subrange thereof) and/or dextrose (ranging from about 0% (w/w) to about 2% (w/w), or any subrange thereof). A sample dilution buffer can also comprise a protein source (ranging from about 0% (w/w) to about 25% (w/w), or any subrange thereof), e.g., bovine serum albumin, human serum albumin, fectal calf serum, goat serum. A sample dilution buffer can also comprise a detergent (ranging from about 0% (w/w) to about 2% (w/w), or any subrange thereof), e.g., Tween-20, Triton X-100, and/or NP-40. A sample dilution buffer can also comprise a denaturant (ranging from about 0 mg/ml to about 5 mg/ml, or any subrange thereof), e.g., urea and/or guanidine hydrochloride. A sample dilution buffer can also comprise a globulin (ranging from about 0 mg/ml to about 5 mg/ml, or any subrange thereof), e.g., mouse IgG, rabbit IgG, goat IgG, rat IgG, bovine gamma, and/or human IgG. A sample dilution buffer can also comprise a heterophilic blocker (ranging from about 0 mg/ml to about 0.5 mg/ml, or any subrange thereof), e.g., HBR, HBR-plus, HAMA blocker, Trublock, and/or Superchemiblock. A sample dilution buffer can comprise one, more or all of the above-described substances. In some embodiments, a sample dilution buffer is a phosphate or Tris buffer having a pH ranging from about pH 7 to about pH 8, and comprises suitable salt(s), sugar(s), protein source(s), detergent(s), denaturant(s), globulin(s), and heterophilic blocker(s), e.g., the salt(s), sugar(s), protein source(s), detergent(s), denaturant(s), globulin(s), and heterophilic blocker(s) described above.

The present kits or systems can be used to quantitatively detect a SARS-CoV-2 antigen in sample from any suitable subject. For example, the subject can be a mammal. In some embodiments, the mammal is a human. In some embodiments, the mammal is non-human mammal, e.g., a non-human primate such as a monkey, a rabbit, or a rodent.

The present kits or systems can comprise any suitable device or a reagent for conducting a single molecule array immunoassay. For example, the present kits or systems can comprise any suitable device or a reagent for conducting a single molecule array immunoassay described and/or claimed U.S. Pat. Nos. and patent publication Nos. 8,222,047, 8,236,574, 9,678,068, 8,415,171, 9,952,237, 9,110,025, US-2016-0123969-A1, and US-2018-0003703-A1, which are expressly incorporated by reference in their entireties herein. In some embodiments, the present kits or systems can comprise the device or a reagent for conducting a single molecule array Simoa immunoassay device, or a reagent to be used in the Simoa immunoassay device.

The present kits or systems can further comprise a reducing agent, e.g., DTT, to disassociate a SARS-CoV-2 antigen, e.g., SARS-CoV-2 N (nucleocapsid) polypeptide, from an antibody in a sample.

In some embodiments, the present kits or systems can be used to quantitatively detect SARS-CoV-2 N (nucleocapsid) polypeptide, or a fragment thereof, in a blood sample, e.g., a plasma, serum, dried blood sample or dried blood spot (DBS) sample. In some embodiments, the present kits or systems can be used to quantitatively detect BARS-CoV-2 N (nucleocapsid) polypeptide, or a fragment thereof, in a saliva sample.

The present kits or systems can further comprise reagent(s) for quantitatively detecting an antibody of the subject to SARS-CoV-2, e.g., a class of IgG antibody of the subject to SARS-CoV-2, or a class of IgG antibody of the subject to SARS-CoV-2 N (nucleocapsid) polypeptide, or a fragment thereof. In some embodiments, the present kits or systems can further comprise reagent(s) for quantitatively detecting an antibody of the subject to SARS-CoV-2 as described and/or claimed in U.S. patent application Ser. No. 17/243,502, which is expressly incorporated by reference in its entirety herein.

The present kits or systems can be used for any suitable purposes or applications. For example, the present kits or systems can be used to aid or facilitate diagnosis, prognosis, risk assessment, stratification and/or treatment monitoring of SARS-CoV-2 infection in a subject, and/or for research and drug/vaccine discovery, and/or development for treating or preventing SARS-CoV-2 infection. In some embodiments, the present kits or systems can be used to assess SARS-CoV-2 infection and/or recovery status in a subject, e.g., among the following: 1) no infection; 2) infection, asymptomatic; 3) infection, pre-symptomatic; 4) infection, symptomatic; or 5) infection, recovered.

### D. EXAMPLES

### Example 1. SARS-Coronavirus-2 Nucleocapsid Protein Measured in Blood Using a Simoa Ultra-Sensitive Immunoassay Differentiates COVID-19 Infection with High Clinical Sensitivity

In this example, SARS-CoV-2 nucleocapsid protein (N-protein) was measured in serum, plasma, and dried blood spots (DBS) via ultrasensitive immunoassay. The exemplary test or assay can be used to differentiate PCR+ from PCR− patients, even if asymptomatic.

In this example, by leveraging the exceptional sensitivity of Single Molecule Array (Simoa) immunoassay technology, we could detect and quantitate SARS-CoV-2 antigen directly in venous blood and capillary blood acquired by commercially available finger-stick collection devices. Here we report the development of a blood-based assay for SARS-CoV-2 N-protein and show detection of clinically significant viral loads in active COVID-19 infections, which avoid swabs and the need to sample nasopharyngeal or nasal fluids. Additionally, we report development of a quantitative serology assay for the detection and quantification of IgG specific to the full-spike antigen of SARS-CoV-2, allowing us to survey clearance of viral antigen with concomitant response of the immune system in longitudinal samples from individual donors.

Abstract

The COVID-19 pandemic continues to have an unprecedented impact on societies and economies worldwide. Despite rapid advances in diagnostic test development and scale-up, there remains an ongoing need for SARS-CoV-2 tests which are highly sensitive, specific, and minimally invasive. Here we report development of a highly sensitive single molecule array (Simoa) immunoassay for the detection of SARS-CoV-2 Nucleocapsid protein (N-protein) in venous and capillary blood. In pre-pandemic and clinical sample sets, the assay has 100% specificity and 97.4% sensitivity for samples collected over two cohorts. The limit of detection (LoD) estimated with viral dilutions is 0.2 pg/ml, corresponding to 0.05 TCID50 per ml, approximately 2200 times more sensitive than an EUA approved antigen test. No cross-reactivity to other common respiratory viruses, including hCoV229E, hCoV OC43, hCoV NL63, Influenza A or Influenza B, was observed. The Simoa SARS-CoV-2 N-protein assay has the potential to detect COVID-19 infection via antigen in blood with similar or better performance characteristics of molecular tests, while also enabling at home and point of care sample collection.

Materials and Methods

Samples. Healthy pre-pandemic serum and plasma samples (collected before December 2019) were obtained from a first source (Source A). Serum and plasma samples from COVID-19 positive donors, as demonstrated by positive RT-PCR test, were obtained from Source A and from a second source (Source B). Samples were collected between Apr. 6 and Jun. 17, 2020. RT-PCR was performed between Mar. 6 and Jun. 12, 2020. Plasma samples from hospitalized COVID-19 patients, as demonstrated by positive RT-PCR test, were provided by a third source (Source C). Samples were collected between Mar. 30 and Apr. 22, 2020. RT-PCR was performed between Mar. 30 and Apr. 15, 2020. Dried blood microsamples were collected using Mitra® Devices (Neoteryx, Torrance, Calif.) from a fourth source (Source D). COVID-19 status of each donor was determined by prior RT-PCR test. Gamma-inactivated SARS-CoV-2 virus was obtained from BEI (beiresources.org), heat-inactivated SARS-CoV-2 and microbial specimens for cross-reactivity testing were obtained from ZeptoMetrix. (zeptometrix.com).

Assay Development. Single Molecule Array (Simoa) technology offers sensitivity up to 1000-fold greater than traditional immunoassays^{16,17}. In brief, the technology involves performing a paramagnetic microbead-based sandwich ELISA, followed by isolation of individual capture beads in arrays of femtoliter-sized reaction wells. Singulation of capture beads within microwells permits buildup of fluorescent product from an enzyme label, so that signal from a single immunocomplex can be detected with a CCD camera in 30 seconds. At very low analyte concentrations, Poisson statistics dictate that bead-containing microwells in the array will contain either a single labeled analyte molecule or no analyte molecules, resulting in a digital signal of either “active” or “inactive” wells. Data collection involves counting active wells corresponding to single enzyme labels. At higher analyte concentrations, digital measurements transition to analog measurements of total fluorescence intensity. Simoa data are reported as Average Enzymes per Bead (AEB). It is widely used in the field of neurodegenerative disease and recently, for the measurement of SARS-CoV-2-associated biomarkers^{18,19}. It has also been demonstrated to rival the sensitivity of PCR for monitoring HIV infection through measurement of the p24 capsid protein in blood^{20,21}.

Simoa N-protein Assay. Antibodies and antigens were obtained from commercial sources. Eight different antibodies and five antigens were screened, resulting in more than 60 different test configurations. The antibody and antigen combination that produced the best signal/background ratio for both calibrator and positive samples was selected. Diluent formulations, detector antibody and Streptavidin-β-Galactosidase concentrations were then optimized, as well as assay protocols (2-step vs 3-step; incubation times). A phosphate-based sample diluent was selected with EDTA to inhibit proteases, heterophilic blocker and a detergent to help de-envelope and inactivate virus particles.

Simoa IgG Assay. A similar assay development approach to that of the N-protein Assay was employed. In this assay configuration, the capture agent is a SARS-CoV-2 spike protein, the detector is a biotinylated Goat anti-Human IgG-Fc Fragment, and the assay is calibrated using a chimeric human/mouse anti-SARS-CoV-2 IgG. (See e.g., U.S. provisional application No. 63/018,465, filed Apr. 30, 2020, and U.S. provisional application No. 63/053,364, filed Jul. 17, 2020) We screened three commercially available spike proteins for the capture molecule and three commercially available anti-human IgG antibodies to select the optimal assay configuration.

Assay Verification. N-protein Assay. The assay was verified by testing 6 runs over 3 days over 2 lots, for a combined total of 12 runs. Precision was determined using 2 diluent-based controls and 3 matrix based spiked samples. Limit of detection (LoD) was determined by diluting gamma-inactivated virus into sample matrix, at varying levels, and admixture linearity was demonstrated using negative matrix spiked with heat-inactivated virus, and then mixed in varying ratios with a separate non-spiked matrix.

IgG Assay. Verification was performed similarly with the exception that the chimeric calibrator antibody was used instead of inactivated virus for LoD and spiking experiments.

Sample Types. We screened samples of serum, K₂EDTA plasma, and dried blood spots. Serum and plasma were diluted 4-fold on the HD-X instrument for the N-protein assay and diluted offline 1000-fold for the IgG assay. Dried blood spots were collected using Mitra collection kits from Neoteryx according to standard protocols (https://www.neoteryx.com/home-blood-blood-collection-kits-dried-capillary-blood), which absorb 20 μl of whole blood. Mitra tips were extracted into 250 μl of sample diluent with shaking at 400 rpm overnight at 2-8° C., resulting in a 12.5-fold sample dilution. All sample results reported have been corrected for dilution factors, to represent the concentration within the sample matrix.

Results and Discussion

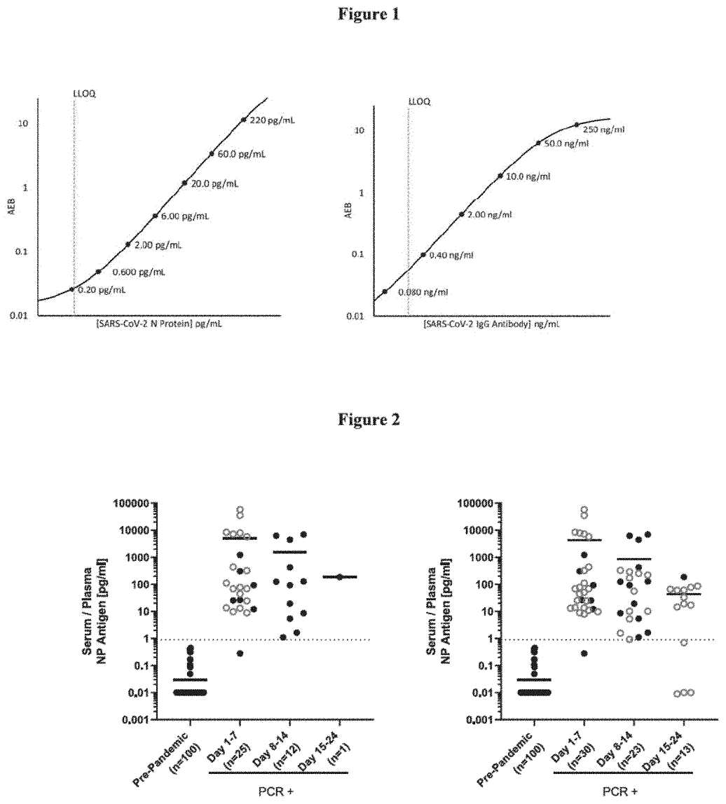

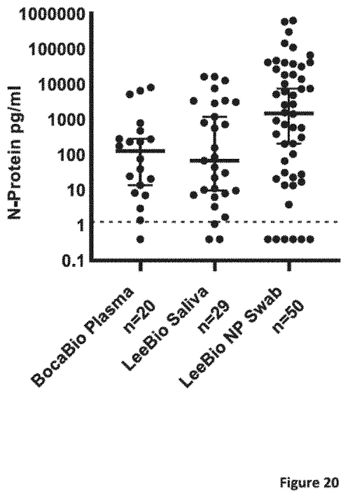

The calibration curves and performance characteristics of both assays are shown in FIG. 1 and Table 1. To determine the clinical utility of the N-protein assay for serum and plasma, we measured PCR+ samples from Source B and the Source C, and pre-pandemic samples from Source A (FIG. 2). We combined serum and K₂EDTA plasma samples in this analysis because we saw excellent correlation between matched serum/K2EDTA plasma from PCR+ donors, suggesting a high degree of matrix equivalency (FIG. 20). FIG. 2 panel A represents only “first-draw” samples, in which every data point represents a unique donor. This use-case is appropriate for a test that is intended to screen novel patients as positive or negative ²². A preliminary clinical cutoff of 0.9 pg/ml (dashed line) for this data set confers a clinical sensitivity of 97.6% (37/38 positives>0.9 pg/ml) and clinical specificity of 100% (100/100 negatives<0.9 pg/ml).

TABLE 1
Performance characteristics of Simoa SARS-CoV-2 N Protein
Advantage Assay and Simoa SARS-CoV-2 IgG Antibody Test.

| | Antigen Assay | IgG Antibody Test |
|---|---|---|
| Minimum Required | 4 × (serum and plasma) | 1000 × (serum, plasma, |
| Dilution (MRD) | 12.5 × (DBS) | and DBS) |
| Required Sample | 25 μl (serum and plasma) | 10 μl |
| Volume | 20 μl (DBS) | |
| Assay Range | 0.9-800 pg/ml (serum | 0.21-250 μg/ml (serum, |
| (adjusted for | and plasma) | plasma, and DBS) |
| dilution) | 2.8-2500 pg/ml (DBS) | |
| Clinical Specificity | 100% | 99% |
| Clinical Sensitivity | 97.6% | 60.3% 0-7 days |
| | | 87.5% for 8-14 days |
| | | 100% for >15 days |
| Limit of Blank | 0.1 pg/ml | 0.029 ng/mL |
| Limit of Detection | 0.32 pg/ml (0.047 | 0.052 ng/mL |
| | TCID50/ml) | |
| Limit of | 0.91 pg/ml (0.094 | 0.213 ng/mL |
| Quantification | TCID50/ml) | |
| Precision | ~6% within-run | 7.0% within-run |
| | ~6% between-run | 2.9% between-run |
| | ~4% between-day | 3.2% between-day |
| Dilution Linearity | ~102% recovery | Average 98.5% |
| Spike Recovery | ~98% | Average 97% |

We binned the samples by day from reported symptom onset or from date of PCR for patients with no reported symptoms for the Source B samples, and day from hospitalization for the Source C cohort. In FIG. 2 panel B we include multiple timepoints from longitudinal donors (Source C) to develop an initial picture of the presence of viral antigen in blood over time. Using an immunoassay for SARS-CoV N-protein, Che et al. observed clinical sensitivity of 94%, 78% and 27% for blood samples within days 1-5, 6-10 and 11-20 of symptom onset¹⁵. Our data shows similar performance, albeit with enhanced sensitivity, notably after the 1st week of infection (FIG. 2 panel B). This shows or suggests the possibility that an ultrasensitive antigen assay could expand the diagnostic window beyond that addressable by the current EUA approved antigen assays that claim clinical sensitivity only within the first 5 to 7 days after onset of symptoms^{10,11}. To determine this, future studies will need to test a sample cohort with well-defined clinical characteristics, in which the onset of infection and symptom are accurately known.

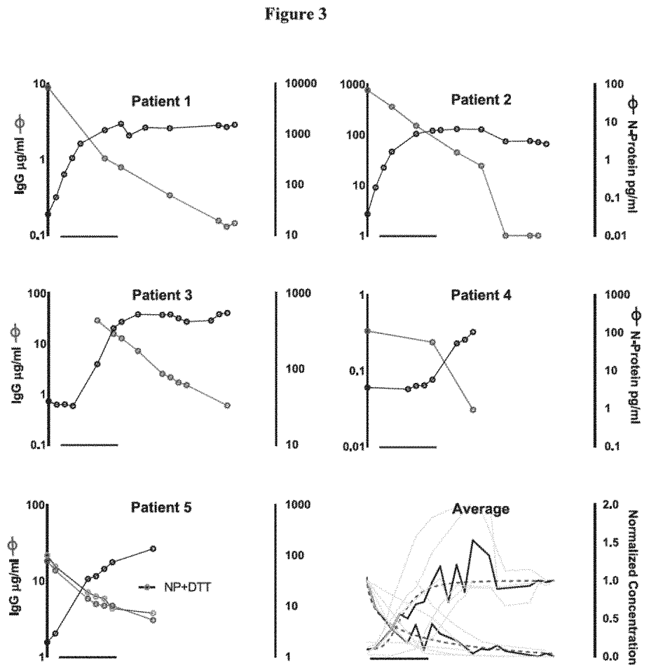

We also measured anti-SAR-CoV-2 specific IgG in the longitudinal samples from the Source C cohort (FIG. 3). N-protein concentration in plasma was observed to decrease over time with a concurrent increase in anti-SARS-CoV2 IgG levels. By normalizing patient responses and using a four-parameter logistic regression to the average response, we find N-protein clearance to occur at 15.6 days and IgG plateau at 7.7 days after hospitalization, and after seroconversion for patients 1, 2 and 5 (FIG. 3). Given that seroconversion for both SARS and SARS-CoV-2 can occur between day 7 to 13 post-symptom^{3,4,14,15,23} we estimate that N-protein clearance occurs near day 22 and IgG plateau near day 14 post-symptom, similar to timelines observed for SARS¹⁵. Ogata et al. observed similar timelines for SARS-CoV-2, although in their study N-protein was generally not detectable once IgG levels had stabilized, whereas our study suggests there is a window of approximately 7 days between IgG plateau and N-protein clearance during which both biomarkers are quantifiable¹⁴.

To determine whether seroconversion and antigen-masking plays a role in the decrease of N-protein signal, we tested samples from patient 5 with and without DTT treatment. We first confirmed that DTT treatment dissociates N-protein-antibody complexes if present, with control experiments using recombinant N-protein and capture antibody spiked into sample matrix (Supplementary FIG. 7). We observed a slight but not significant increase in N-protein levels in samples treated with DTT (FIG. 3 patient 5, gray line) compared to untreated (red line). We thus conclude that decrease in N-protein levels is due to clearance from the blood stream and not masking due to seroconversion.

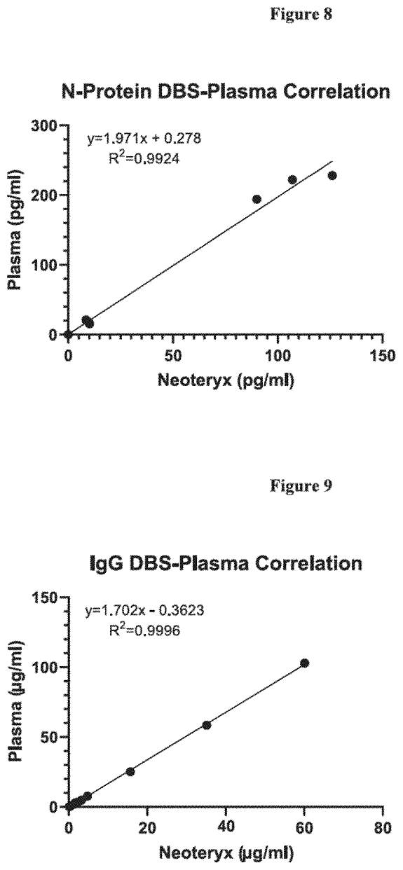

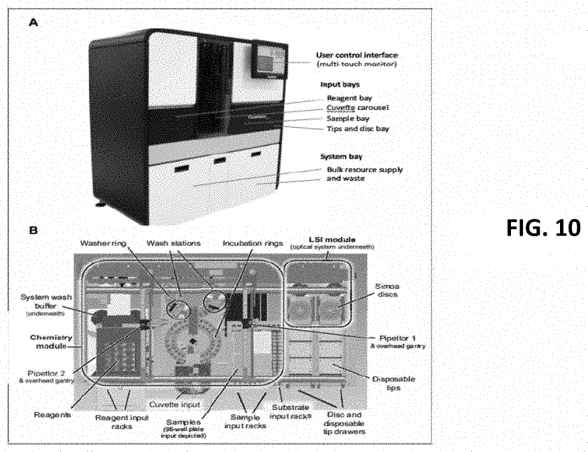

To allow at-home or point-of-care collection of blood samples, we tested dried blood spots (DBS) collected with Mitra® tips (Neoteryx.com). These devices absorb 20 μl of capillary blood from a finger-stick, and users may subsequently store and ship them without cold-chain requirements. To verify the recovery of N-protein from the Mitra tips, we spiked whole blood with recombinant N-protein and then processed it either into plasma or DBS. We then measured N-protein in both sample types, which showed excellent correlation of 0.993 R². We performed this same correlation experiment for SARS-CoV-2 specific IgG antibody (Supplementary FIGS. 8 and 9).

We measured N-protein levels in DBS patient samples collected in the presence of an active COVID-19 infections using the Mitra devices (SOURCE D cohort). Source D has established a practice of testing residents and staff for COVID-19 weekly using an authorized molecular test. In this study, DBS samples were collected at two time points, one week apart, for measurement of N-protein and IgG levels by Simoa. Table 2 summarizes sample collection and testing dates for this study cohort across all relevant assays. This enabled a comparison of the performance of the Simoa SARS-CoV-2 N-Protein Assay against the gold-standard of PCR in the presence of active COVID-19 infections.

TABLE 2
Sampling and testing timeline in SOURCE D study

| | Day 1* | Day 5 | Day 8** | Day 12 |
|---|---|---|---|---|
| PCR test | 20 donors | | 22 donors total | |
| | | | 4 donors died, 1 declined | |
| | | | 7 new donors enrolled | |
| DBS | | 20 donors | | 22 donors |
| collection | | | | |

*PCR for two donors done on Day −2 and three donors on Day −1.
**PCR for one donor on Day −5, one on Day 2 and one on Day 12.

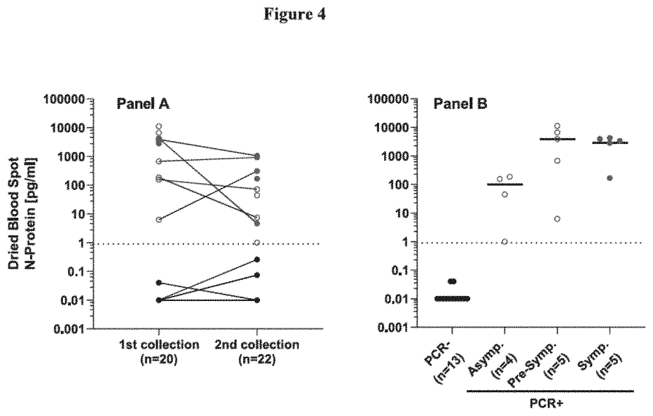

In FIG. 4 panel A we show N-protein levels measured in DBS samples for two timepoints, where connecting lines between points denote changes in individual donor levels from week 1 to week 2. Black symbols indicate PCR− donors and red symbols PCR+. Open red symbols indicate PCR+ donors that exhibited no symptoms at time of collection (either asymptomatic or pre-symptomatic). This data demonstrates 100% sensitivity and specificity of the Simoa N-protein assay compared to PCR, and notably the Simoa N-protein assay identified COVID-19 positive status for four donors that exhibited no symptoms over the course of infection (asymptomatic) and five donors that developed symptoms after sample collection (pre-symptomatic). The time course of one pre-symptomatic donor in particular illustrated the ability of N-Protein in capillary blood to diagnose COVID-19 before symptom onset: Donor 12 enrolled nominally as a negative control and tested PCR− on day 1; DBS sampled on day 5 showed elevated levels of N-Protein (first collection) before symptom onset; confirmed positive with PCR testing on day 7; symptoms developed on day 8; by day 15 the donor had recovered. Donor 12 may represent a false-negative PCR result that was flagged by the N-protein assay, although we cannot be sure since the PCR test and DBS collection were five days apart. Future studies will aim to address this question through direct comparison of clinical sensitivity of PCR and the Simoa SARS-CoV-2 N-Protein assay on samples collected concurrently.

False negative PCR results represent a significant challenge in the COVID-19 pandemic⁵. Kucirka et al. report the highest probability of PCR false-negative results before symptom onset, with the false-positive rate decreasing from 100% to 67% in the first four days post-infection. On day 5, the median time for symptom-onset, the probably was still 38%^{6,24}. Compounding the problem of poor sensitivity in pre-symptomatic patients, He et al. observed the highest viral load in throat swabs at time of symptom onset, and inferred that infectiousness will peak on or before symptom onset²⁵. In this context, the ability of the Simoa SARS-CoV-2 N-Protein assay to detect pre-symptomatic individuals will be particularly important.

In the SOURCE D cohort, 8 of the 14 PCR+ donors presented without symptoms despite testing PCR and N-protein positive, suggesting that virus antigen is present prior to symptom onset. In FIG. 4 panel B, we separated donors into four groups: PCR−; asymptomatic PCR+, that did not show symptoms at any point during infection; pre-symptomatic PCR+, that did not show symptoms at time of collection but developed symptoms by the day of the second collection; and symptomatic PCR+, that presented with symptoms at the first collection. Fully asymptomatic donors have a lower median level of N-Protein, however we do not see different levels of N-protein in pre-symptomatic or symptomatic donors, suggesting that antigen presents in capillary blood at high levels before symptom onset. Ogata et al. suggested that viral antigen would only transfer to blood in severe or late-stage cases, however our data suggest that some mechanism exists for viral antigen to transfer without severe infection¹⁴. Che et al. reported similar trends for SARS-CoV patients, who had a higher positive detection rate of N-protein in serum samples within the first 10 days of infection than that detected by RT-PCR in respiratory samples, an observation hypothesized to be associated in part with respiratory specimen collection variables leading to false negatives¹⁵.

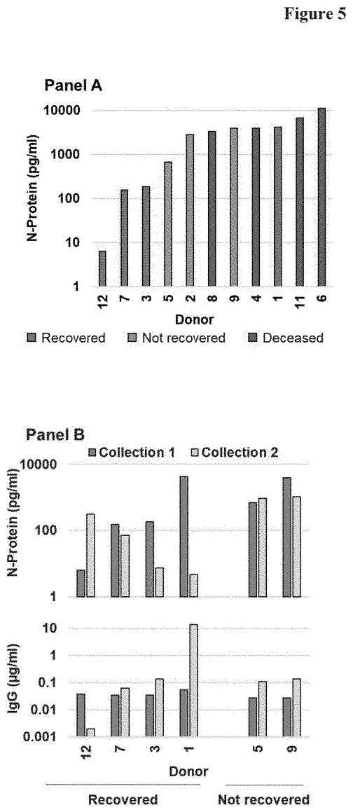

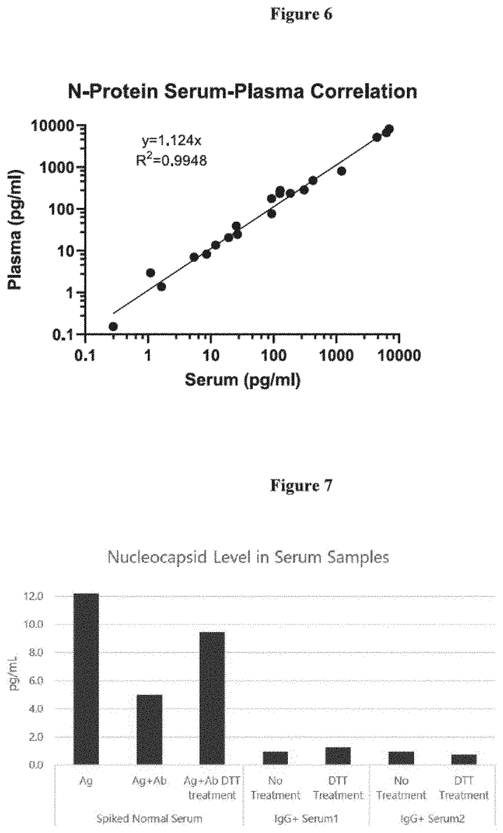

Ogata et al. observed a correlation between SARS-CoV-2 antigen levels and disease severity, looking at the indicators of fraction of patients admitted to the ICU, patients intubated and days between hospitalization and intubation¹⁴. Similarly, we observed that higher concentrations of N protein appear to be associated with greater disease severity, and clearance of the antigen associated with greater recoverability. In FIG. 5 panel A we have ranked SOURCE D PCR+ donors by N-protein level, and color-coded results according to disease outcome: deceased; not recovered at collection 2; or recovered at collection 2. In this limited sample set, we observe a trend of worse clinical outcome associated with higher N-protein level. In FIG. 5 panel B, we have grouped donors into recovered (n=4) and not recovered (n=2) and display N-protein levels for both collections. Average N-protein level decreases 10-fold (1143 to 98 pg/ml) for recovered donors across both collection dates, contrasted with a higher starting average and more moderate decrease of 2-fold (2287 to 988 pg/ml) for not recovered donors (FIG. 5B panel B).

We measured SARS-CoV-2 specific IgG in the SOURCE D cohort and found low IgG levels for all donors at collection 1, suggesting that seroconversion had not yet occurred. At collection 2 we observed slight increases in IgG for some donors, but a large increase only for donor 1 (FIG. 5 panel B lower half). This donor saw a concomitant, large decrease in N-protein and was the only donor with very high N-protein levels to recover by the 2nd collection (FIG. 5 panel B upper half). Table 3 below details the timelines, N-protein levels, and IgG levels for all SOURCE D samples measured.

TABLE 3
N-protein and SARS-CoV-2 specific IgG concentrations measured in the SOURCE D
cohort for all donors and both collections.

| | Collection Date: Jul. 20, 2020 | | | | Collection Date: Jul. 27, 2020 | | | |
|---|---|---|---|---|---|---|---|---|
| | days | days | | | days | days | | |
| Donor | from | from | | | from | from | NP | |
| ID | symptom | PCR | NP pg/ml | Spk IgG | symptom | last PCR | pg/ml | Spk IgG |
| 1 | 7 | 7 | 4226.08 | 0.06 | 14 | 14 | 4.62 | 13.41 |
| 2 | 8 | 7 | 2811.23 | 0.00 | intubated | | | |
| 3 | 6 | 6 | 186.14 | 0.03 | 13 | 13 | 7.41 | 0.14 |
| 4 | 9 | 6 | 3933.59 | 0.01 | died | | | |
| 5 | | 6 | 677.47 | 0.03 | | 13 | 925 | 0.11 |
| 6 | | 5 | 11235.56 | 0.01 | died | | | |
| 7 | | 5 | 154.47 | 0.03 | | 12 | 72.2 | 0.06 |
| 8 | 4 | 5 | 3349.23 | 0.01 | Opted out of study --> died | | | |
| 9 | | 5 | 3896.05 | 0.03 | | 12 | 1051 | 0.14 |
| 11 | | 5 | 6658.056 | 0.208 | died | | | |
| 12 | −3 | 5 | 6.260 | 0.037 | 4 | 4 | 308 | 0.002 |
| 13 | | 5 | 0.010 | 0.001 | | 12 | 0.01 | 0.005 |
| 14 | | 5 | 0.010 | 0.014 | | 4 | 0.01 | 0.002 |
| 15 | | 5 | 0.040 | 0.024 | | 4 | 0.01 | 0.021 |
| 16 | | 5 | 0.010 | 0.011 | | 4 | 0.260 | 0.015 |
| 17 | | 5 | 0.040 | 0.005 | | 4 | 0.01 | 0.013 |
| 18 | | 5 | 0.010 | 0.000 | | 4 | 0.074 | 0.000 |
| 19 | | 5 | 0.010 | 0.019 | | 4 | 0.01 | 0.012 |
| 20 | | 5 | 0.010 | 0.024 | | 4 | 0.01 | 0.009 |
| 21 | | 5 | 0.010 | 0.012 | | 4 | 0.074 | 0.011 |
| 22 | | | | | | 4 | 0.01 | 0.005 |
| 23 | | | | | | 17 | 0.993 | 0.702 |
| 24 | | | | | | 4 | 44.4 | 0.002 |
| 25 | | | | | | 4 | 0.01 | 0.031 |
| 26 | | | | | 0 | 0 | 167 | 0.003 |
| 27 | | | | | | 11 | 0.01 | 0.054 |
| 28 | | | | | | 4 | 0.01 | 0.144 |

Conclusion

In summary, we have developed a blood-based assay for SARS-CoV-2 N-Protein and shown detection of clinically significant viral loads in active COVID-19 infections, using sample collection methods that avoid swabs and the need to sample nasopharyngeal or nasal fluids. We estimate a clinical sensitivity of 97.4% in serum/plasma using two PCR+ cohorts and clinical specificity of 100% using a cohort of 100 pre-pandemic samples. We see no cross-reactivity to other common respiratory viruses, including hCoV229E, hCoVOC43, hCoVNL63, Influenza A or Influenza B (Table 4 below). Using titers of gamma-inactivated virus we estimate the limit of detection (LoD) of our assay to be 0.05 TCID50, at least 2200 times more sensitive than current antigen tests with EUA approval for use in nasal swabs¹⁰.

TABLE 4
Inactivated, cultured virus was purchased from Zeptometrix, and tested
for cross-reactivity at the TCID₅₀ levels listed. No cross-reactivity was
observed.

| | | | Titer | Conc | |
|---|---|---|---|---|---|
| | | | Tested | Measured by | |
| | | | | N antigen | |
| Virus | | | TCID₅₀/ | Assay | |
| Description | Vendor | Cat# | mL | Serum | Plasma |
| Adenovirus | Zeptometrix | 0810021CFHI | 3.52E+04 | <LoD | <LoD |
| Type 07 | | | | | |
| (Species B) | | | | | |
| Culture | | | | | |
| Fluid | | | | | |
| Enterovirus | Zeptometrix | 0810237CFHI | 3.78E+05 | <LoD | <LoD |
| Type 68 | | | | | |
| (2007 | | | | | |
| Isolate) | | | | | |
| Culture | | | | | |
| Fluid | | | | | |
| Influenza A | Zeptometrix | 0810036CFHI | 2.88E+05 | <LoD | <LoD |
| H1N1 | | | | | |
| (New | | | | | |
| Cal/20/99) | | | | | |
| Culture | | | | | |
| Fluid | | | | | |
| Influenza B | Zeptometrix | 0810037CFHI | 3.52E+04 | <LoD | <LoD |
| (Florida/02/ | | | | | |
| 06) Culture | | | | | |
| Fluid | | | | | |
| Parainfluenza | Zeptometrix | 0810014CFHI | 2.28E+06 | <LoD | <LoD |
| Virus Type | | | | | |
| 1 Culture | | | | | |
| Fluid | | | | | |
| Parainfluenza | Zeptometrix | 0810015CFHI | 2.88E+05 | <LoD | <LoD |
| Virus Type | | | | | |
| 2 Culture | | | | | |
| Fluid | | | | | |
| Parainfluenza | Zeptometrix | 0810016CFHI | 1.65E+06 | <LoD | <LoD |
| Virus Type | | | | | |
| 3 Culture | | | | | |
| Fluid | | | | | |
| Parainfluenza | Zeptometrix | 0810060CFHI | 7.05E+05 | <LoD | <LoD |
| Virus Type | | | | | |
| 4A Culture | | | | | |
| Fluid | | | | | |
| Respiratory | Zeptometrix | 0810040ACFHI | 9.50E+05 | <LoD | <LoD |
| Syncytial | | | | | |
| Virus Type | | | | | |
| A (Isolate: | | | | | |
| 2006 | | | | | |
| Isolate) | | | | | |
| Culture | | | | | |
| Fluid | | | | | |
| Rhinovirus | Zeptometrix | 0810012CFNHI | 8.88E+04 | <LoD | <LoD |
| Type 1A | | | | | |
| Culture | | | | | |
| Fluid | | | | | |
| Coronavirus | Zeptometrix | 0810229CFHI | 1.04E+05 | <LoD | <LoD |
| (Strain: | | | | | |
| 229E) | | | | | |
| Culture | | | | | |
| Fluid | | | | | |
| Coronavirus | Zeptometrix | 0810024CFHI | 2.63E+05 | <LoD | <LoD |
| (Strain: | | | | | |
| OC43) | | | | | |
| Culture | | | | | |
| Fluid | | | | | |
| Coronavirus | Zeptometrix | 0810228CFHI | 4.25E+04 | <LoD | <LoD |
| (Strain: | | | | | |
| NL63) | | | | | |
| Culture | | | | | |
| Fluid | | | | | |

We have also developed a quantitative serology assay for the detection and quantification of IgG specific to the full-spike antigen of SARS-CoV-2, allowing us to survey clearance of viral antigen with concomitant response of the immune system in longitudinal samples from individual donors. We have demonstrated detection in capillary blood using the Neoteryx Mitra® dried blood spot (DBS) collection device, which enables at-home and point-of-care sample collection. Using DBS samples, we successfully monitored disease status of staff and residents during an active SARS-CoV-2 outbreak, with the ability to report positivity with sensitivity equal to or greater than molecular testing.

We plan further studies to validate the ability of the SARS-CoV-2 N-Protein assay to diagnose COVID-19 and determine if it has comparable or better sensitivity than molecular testing. Larger cohorts are needed with better characterized clinical symptoms and timelines. In particular, cohorts with well-defined onset of infection are needed to determine the window of effectiveness of the SARS-CoV-2 N-Protein assay, which may be able to diagnose both earlier (pre-symptomatic infection) than molecular testing and later (beyond one week post-symptom) than currently EUA approved antigen tests.

The SARS-CoV-2 antigen assay has the potential to be available for widespread deployment through minimally invasive remote and home sample collection. In one embodiment, this SARS-CoV-2 antigen assay may provide a new, orthogonal method for early detection of SARS-CoV-2 infection to augment the accuracy and availability of the SARS-CoV-2 testing arsenal.

### REFERENCES

Cited references are listed below.
1. Max Roser Hannah Ritchie, E. O.-O. & Hasell, J. Coronavirus Pandemic (COVID-19). Our World Data (2020).
2. U.S. Food & Drug Administration. Coronavirus (COVID-19) Update: FDA Authorizes First Antigen Test to Help in the Rapid Detection of the Virus that Causes COVID-19 in Patients [press release]. (2020).
3. Amanat, F. et al. A serological assay to detect SARS-CoV-2 seroconversion in humans. Nat. Med. 26, 1033-1036 (2020).
4. Norman, M. et al. Ultra-Sensitive High-Resolution Profiling of Anti-SARS-CoV-2 Antibodies for Detecting Early Seroconversion in COVID-19 Patients. medRxiv: the preprint server for health sciences (2020) doi:10.1101/2020.04.28.20083691.
5. Woloshin, S., Patel, N. & Kesselheim, A. S. False Negative Tests for SARS-CoV-2 Infection—Challenges and Implications. N. Engl. J. Med. (2020) doi:10.1056/NEJMp2015897.
6. Kucirka, L. M., Lauer, S. A., Laeyendecker, O., Boon, D. & Lessler, J. Variation in False-Negative Rate of Reverse Transcriptase Polymerase Chain Reaction-Based SARS-CoV-2 Tests by Time Since Exposure. Ann. Intern. Med. (2020) doi:10.7326/M20-1495.
7. Fajnzylber, J. M. et al. SARS-CoV-2 Viral Load is Associated with Increased Disease Severity and Mortality. medRxiv 2020.07.15.20131789 (2020) doi:10.1101/2020.07.15.20131789.
8. Scohy, A. et al. Low performance of rapid antigen detection test as frontline testing for COVID-19 diagnosis. J. Clin. Virol. Off. Publ. Pan Am. Soc. Clin. Virol. 129, 104455 (2020).
9. Blairon, L., Wilmet, A., Beukinga, I. & Tre-Hardy, M. Implementation of rapid SARS-CoV-2 antigenic testing in a laboratory without access to molecular methods: Experiences of a general hospital. J. Clin. Virol. Off. Publ. Pan Am. Soc. Clin. Virol. 129, 104472 (2020).
10. Quidel. Sofia SARS Antigen FIA—Package Insert. (2020).
11. BD Biosciences. BD Veritor System for Rapid Detection of SARS-CoV-2. (2020).
12. Varga, Z. et al. Endothelial cell infection and endotheliitis in COVID-19. Lancet (London, England) vol. 395 1417-1418 (2020).
13. Wölfel, R. et al. Virological assessment of hospitalized patients with COVID-2019. Nature 581, 465-469 (2020).
14. Ogata, A. F. et al. Serial Profiling of SARS-CoV-2 Antigens and Antibodies in COVID-19 Patient Plasma. medRxiv 2020.07.20.20156372 (2020) doi:10.1101/2020.07.20.20156372.
15. Che, X.-Y. et al. Nucleocapsid protein as early diagnostic marker for SARS. Emerg. Infect. Dis. 10, 1947-1949 (2004).
16. Rissin, D. M. et al. Single-molecule enzyme-linked immunosorbent assay detects serum proteins at subfemtomolar concentrations. Nat. Biotechnol. 28, 595-599 (2010).
17. Wilson, D. H. et al. The Simoa HD-1 Analyzer: A Novel Fully Automated Digital Immunoassay Analyzer with Single-Molecule Sensitivity and Multiplexing. J. Lab. Autom. 21, 533-547 (2016).
18. Kanberg, N. et al. Neurochemical evidence of astrocytic and neuronal injury commonly found in COVID-19. Neurology (2020) doi:10.1212/WNL.0000000000010111.
19. Ameres, M. et al. Association of neuronal injury blood marker neurofilament light chain with mild-to-moderate COVID-19. Journal of neurology 1-3 (2020) doi:10.1007/s00415-020-10050-y.
20. Chang, L. et al. Simple diffusion-constrained immunoassay for p24 protein with the sensitivity of nucleic acid amplification for detecting acute HIV infection. J. Virol. Methods 188, 153-160 (2013).
21. Cabrera, C., Chang, L., Stone, M., Busch, M. & Wilson, D. H. Rapid, Fully Automated Digital Immunoassay for p24 Protein with the Sensitivity of Nucleic Acid Amplification for Detecting Acute HIV Infection. Clin. Chem. 61, 1372-1380 (2015).
22. U.S. Food & Drug Administration. Antigen Template for Manufacturers (May 11, 2020). (2020).
23. Long, Q.-X. et al. Antibody responses to SARS-CoV-2 in patients with COVID-19. Nat. Med. 26, 845-848 (2020).
24. Lauer, S. A. et al. The Incubation Period of Coronavirus Disease 2019 (COVID-19) From Publicly Reported Confirmed Cases: Estimation and Application. Ann. Intern. Med. 172, 577-582 (2020).
25. He, X. et al. Temporal dynamics in viral shedding and transmissibility of COVID-19. Nat. Med. 26, 672-675 (2020).

### Example 2. N-Protein Presents Early in Blood, Dried Blood and Saliva During Asymptomatic and Symptomatic SARS-CoV-2 Infection

Abstract

The COVID-19 pandemic continues to have an unprecedented impact on societies and economies worldwide. There remains an ongoing need for high-performance SARS-CoV-2 tests which may be broadly deployed for infection monitoring. Here we report a highly sensitive single molecule array (Simoa) immunoassay in development for detection of SARS-CoV-2 Nucleocapsid protein (N-protein) in venous and capillary blood and saliva. In all matrices in the studies conducted to date we observe>98% negative percent agreement and >90% positive percent agreement with molecular testing for days 1-7 in symptomatic, asymptomatic, and pre-symptomatic PCR+ individuals. N-protein load decreases as anti-SARS-CoV-2 Spike-IgG increases, and N-protein levels correlate with RT-PCR Ct-values in saliva, and between matched saliva and capillary blood samples. This Simoa SARS-CoV-2 N-protein assay effectively detects SARS-CoV-2 infection via measurement of antigen levels in blood or saliva, using non-invasive, swab-independent collection methods, offering potential for at home and point of care sample collection. The content of the Example 2 is also disclosed in Shan, D., Johnson, J. M., Fernandes, S. C. et al. N-protein presents early in blood, dried blood and saliva during asymptomatic and symptomatic SARS-CoV-2 infection. Nat Commun 12, 1931 (2021). https://doi.org/10.1038/s41467-021-22072-9, which is incorporated herein by reference in its entirety for all purposes.

Introduction

In November 2019, the first cases of SARS-CoV-2 (severe acute respiratory syndrome coronavirus-2) were reported in Wuhan, China and since has caused a worldwide pandemic¹. Molecular testing for viral RNA is the primary diagnostic modality for active infection, while serological tests measure anti-SARS-CoV2 antibodies post-infection^{2,3}. Although RT-PCR-based molecular testing for viral RNA in respiratory specimens is the primary diagnostic tool for active infection, concerns have been raised about the risk of false negative results associated with the use of nasal and nasopharyngeal (NP) swabs⁴, especially before symptom onset. Kucirka et al. estimate probability of a false negative result to decrease from 100% on day 1 post-infection to 67% on day 4. On day 5, the median time for symptom onset, molecular tests still had a 38% probability of producing a false negative result and declined no further than 20% in the days that followed, when the infection should be most detectable⁵. Furthermore, the complexity, significant supply chain challenges, and relatively low throughput of RT-PCR are contributing to the difficulties in developing sufficiently large-scale testing required to enable societies to re-open⁶, prompting a search for additional diagnostic modalities.

Antigen detection by immunoassay offers a simpler workflow and a supply chain diversified from PCR. Prior to January 2021, several SARS-CoV-2 antigen tests were approved by the USFDA for use with nasopharyngeal or nasal swabs. These assays claim positive percent agreement (PPA) with PCR ranging from with 84% to 97.6%⁷. More recently, a Simoa SARS-CoV-2 N Protein Antigen Test for NP swabs with 97.7% PPA received US FDA Emergency Use Authorization⁸, indicating that highly sensitive and specific antigen testing is possible with this technology.

Detection of SARS-CoV-2 antigen in matrices beyond nasal and NP swabs may be of scientific and clinical significance⁹, indeed EUA applications have been approved for molecular testing of SARS-CoV-2 in saliva, which allows easier sample collection and may have better sensitivity than swab-based approaches¹⁰. Furthermore, multiple clinical manifestations suggest that this respiratory virus can migrate from the lungs into the bloodstream. Mehra et al. described evidence of SARS-CoV-2 peripheral involvement during post-mortem histological examination of effected tissues, including electron microscopy images of viral inclusion structures in endothelial cells¹¹. It was hypothesized that SARS-CoV-2 infection may facilitate the induction of endothelitis in multiple organs as a direct consequence of viral involvement. Wölfel et al. reported that SARS-Cov-2 virus was not detectable in blood using molecular diagnostic techniques¹², but additional later studies have found evidence that plasma viremia may play a significant role in disease course and that viral loads in plasma may predict risk of death¹³⁻¹⁵.

In this work, we describe development of a SARS-CoV-2 antigen test using Simoa technology to quantify N-protein in serum/plasma, dried blood microsamples (DBS) and saliva. The assay was designed to target the SARS-CoV-2 Nucleocapsid protein, due to the large copy number per viral particle (˜1000)¹⁶, and due to reports of large numbers of mutations in the SARS-CoV-2 spike protein″. We quantitate SARS-CoV-2 N-protein and anti-SARS-CoV-2 spike IgG directly in multiple sample matrices including serum and plasma from venous collection, capillary blood acquired by finger-stick dried blood microsampling devices (DBS), and saliva. Compared to molecular testing, we observe>90% positive percent agreement (PPA) of SARS-CoV-2 positive patients and >98% negative percent agreement (NPA) in all matrices within 7 days of positive PCR test, both for asymptomatic and symptomatic patients, with the developmental/research assay described herein. An inverse relationship between N-protein and anti-SARS-CoV-2 spike protein IgG is observed, with antigen clearing as IgG increases. In longitudinal saliva and DBS samples, N-protein levels correlate between sample types and with Ct-values measured in saliva. N-protein levels in saliva are higher but more variable than levels in capillary blood. The Simoa N-protein antigen test represents a robust SARS-CoV-2 detection tool in multiple types of sample matrix.

Results

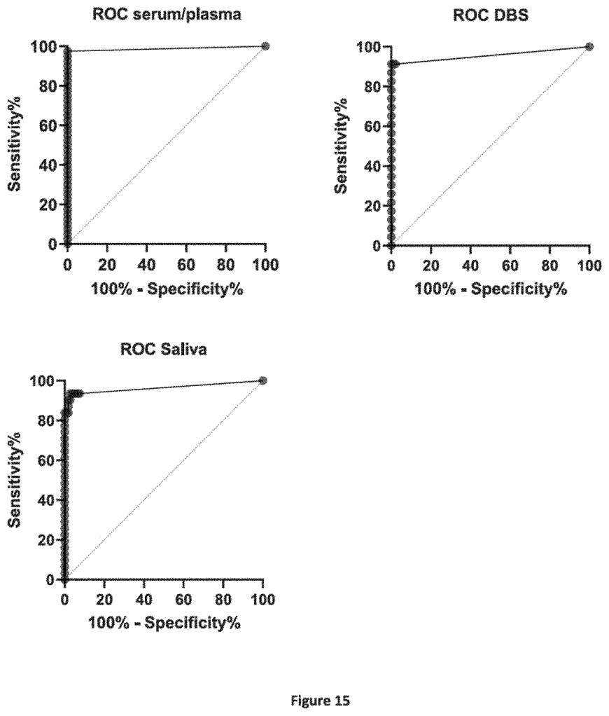

ROC analysis and cutoff. We established preliminary cutoffs via ROC analysis for all sample types, as detailed in Methods in Supplementary Information. ROC curves are shown in FIG. 15. Positive/negative cutoffs were determined to be the greater of either the functional limit of quantification (fLoQ) of the assay or the Youden-Index recommended value. In all matrices, the cutoff was determined to be the fLoQ, as described in the assay data sheet (see Methods). The cutoff for SARS-CoV-2 spike IgG was as determined as per the EUA authorized test (see Methods). Dashed lines in figures represent the relevant positive/negative cutoff for each matrix. We consider these cutoffs as preliminary and acknowledge that they may change upon further studies.

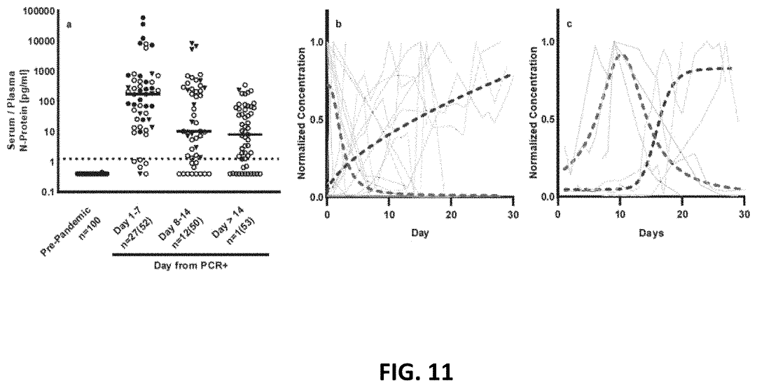

Serum and plasma samples. We measured N-protein in pre-pandemic sera (n=100), in SARS-CoV-2 RT-PCR+ samples from a commercial source (n=20) and in longitudinal plasma from the University of Bonn (n=20 donors, total of n=135 longitudinal samples). N-protein levels in each sample are shown binned by days from PCR in FIG. 11. With U.Bonn donors PCR testing was performed and sample collection commenced on the first day of hospitalization; the date of symptom onset was not available for this cohort. Applying a preliminary cutoff of 1.25 pg/mL N-protein (FIG. 11A, dashed line), indicated assay NPA of 100% and PPA of 97.5% in first-draw samples, independent of number of days from PCR. Although most longitudinal samples remained positive for N-protein>14 days following initial PCR test (88% day 1-7, 78% day 8-14 and 72% day>14), a downward trend in N-protein concentrations over time was observed for individual donors.

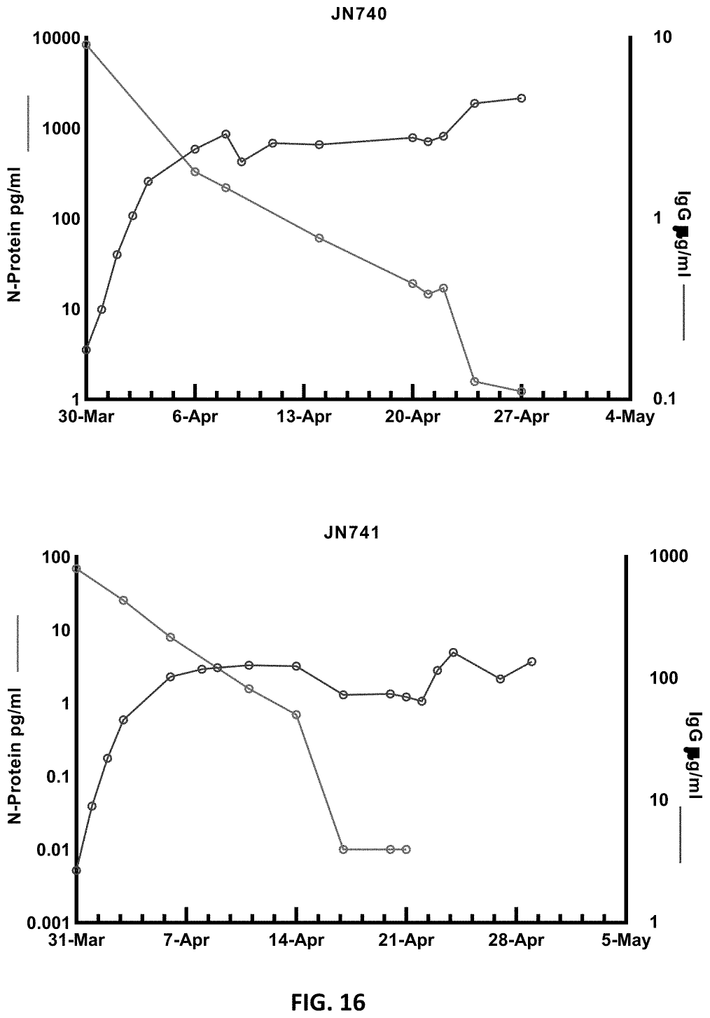

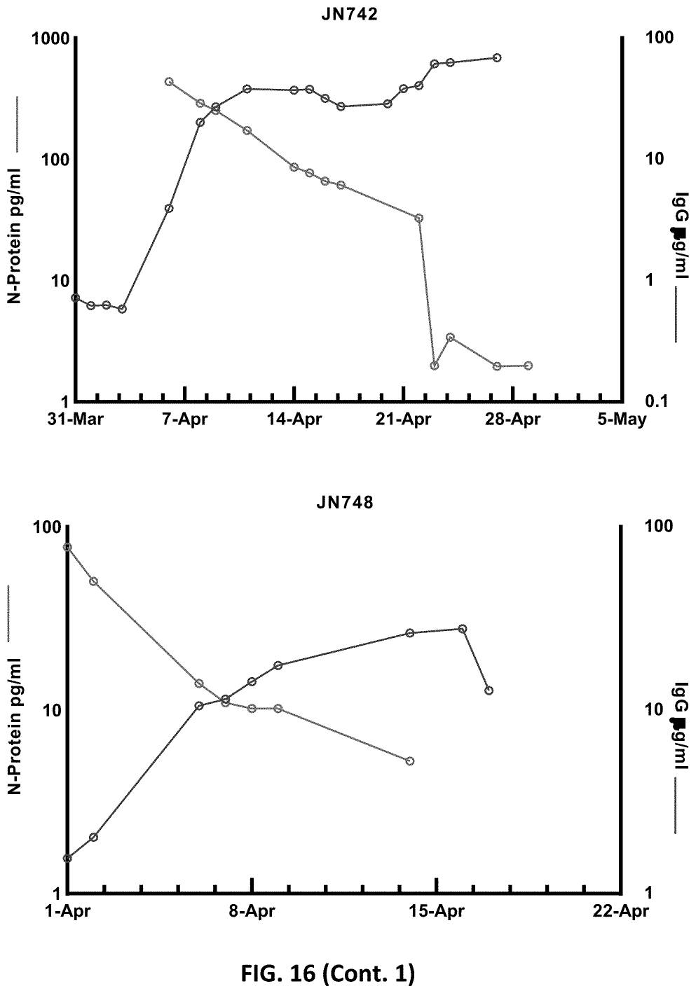

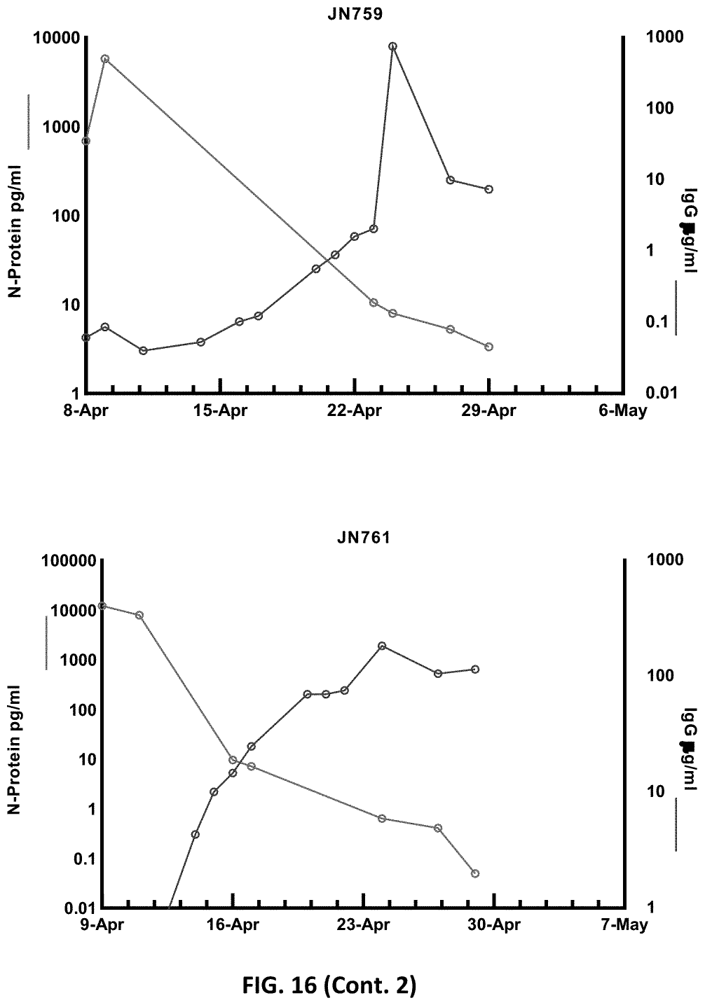

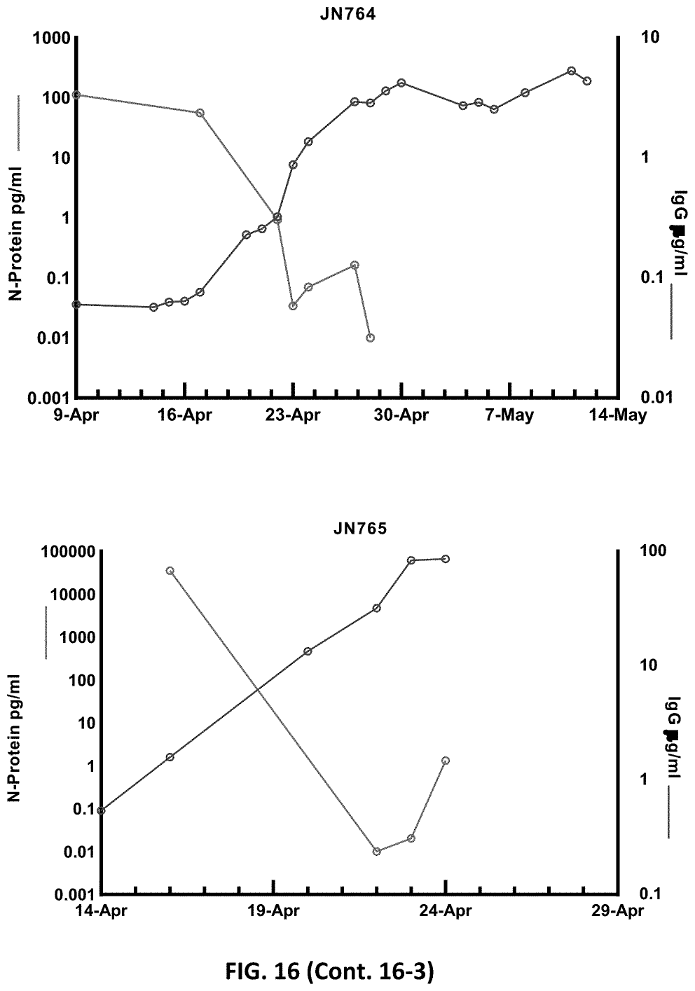

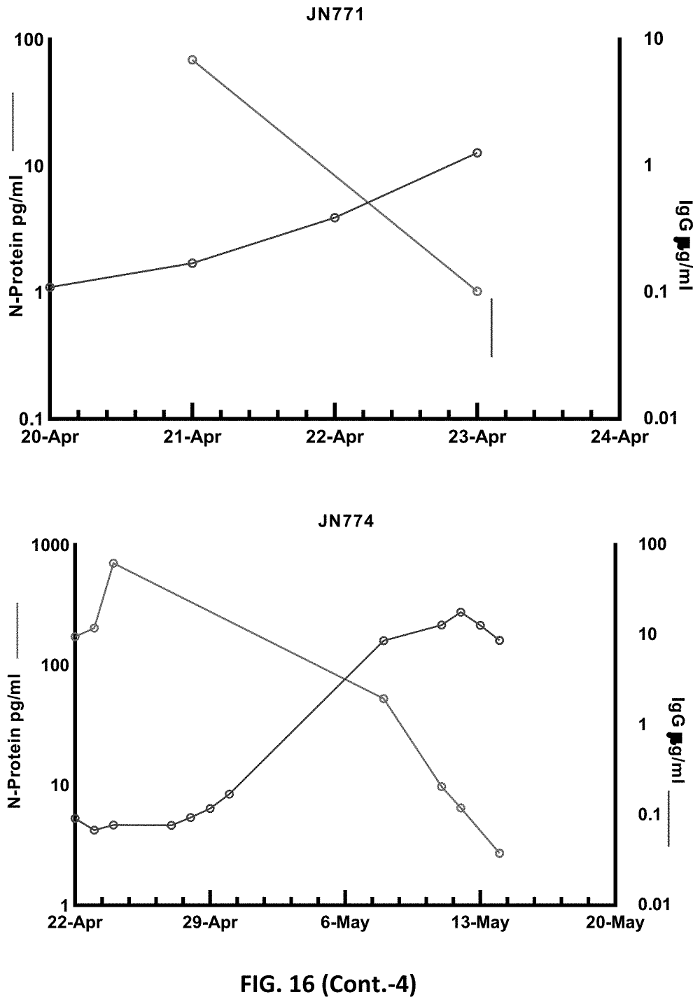

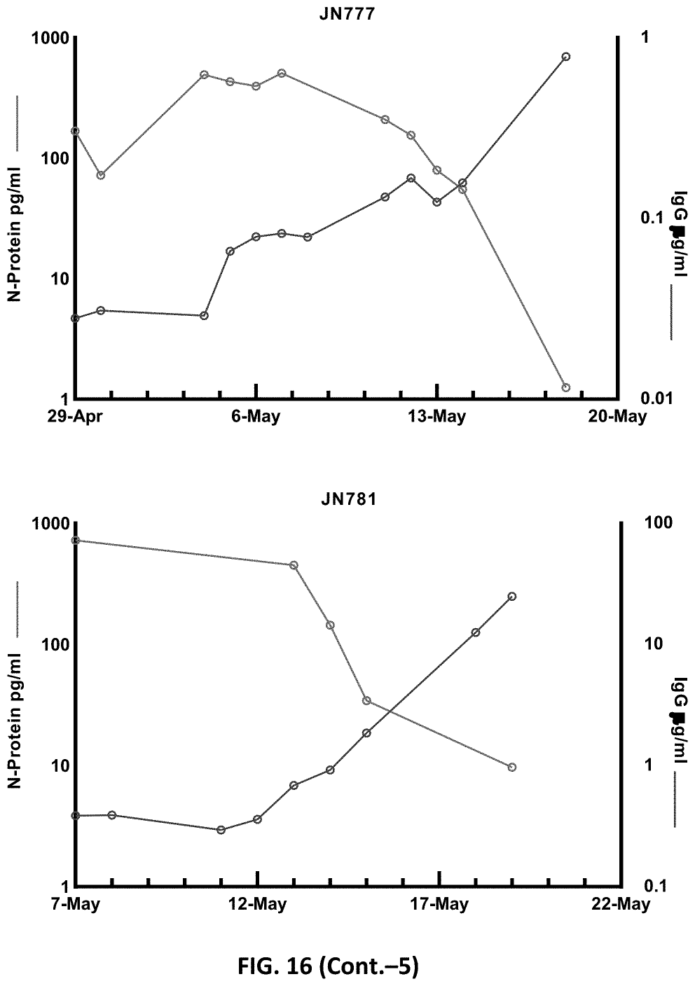

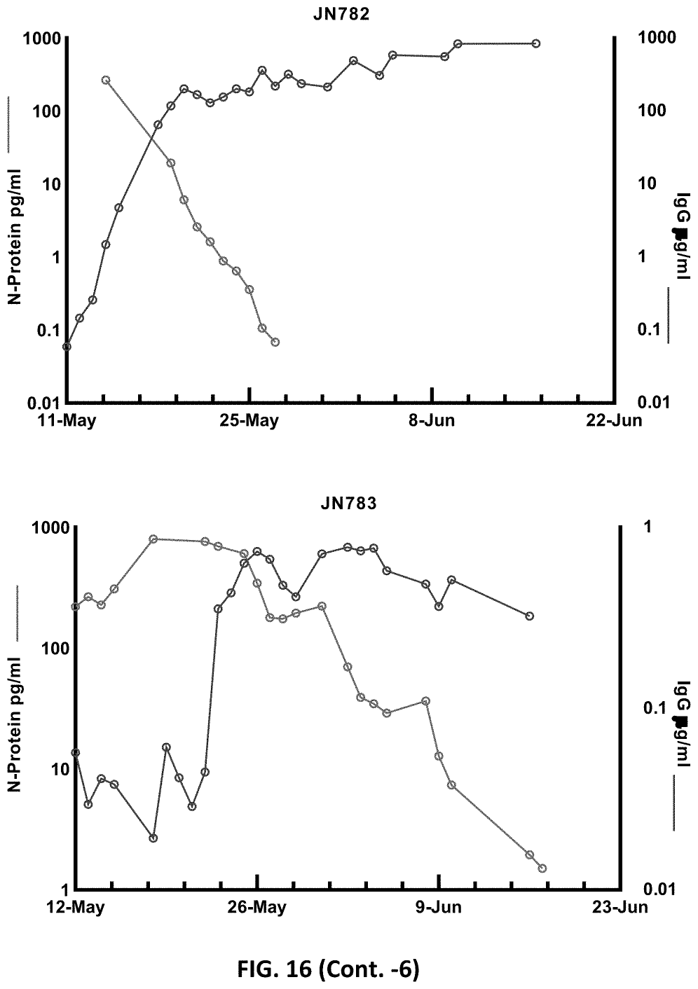

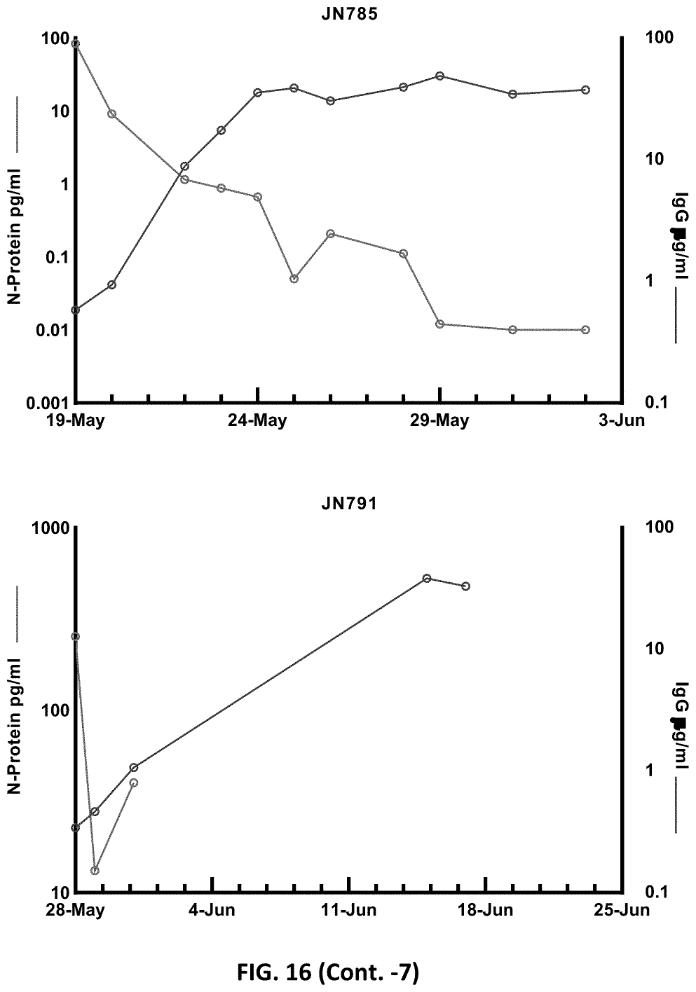

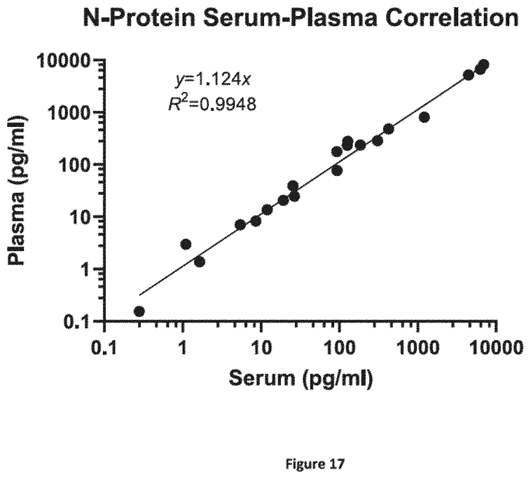

Within the longitudinal cohort we also measured anti-SARS-CoV-2 spike protein IgG for 141 timepoints over 16 patients (FIG. 16). As N-Protein decreased over time, we observed a concurrent increase in IgG. To explore the kinetics of N-protein levels relative to the serological response, we separated data sets into those with N-protein maximum concentration at day 1 (FIG. 11B; n=10) and after day 1 (FIG. 11C; n=3), normalized to maximum response and aligned to peak N-Protein levels. Using non-linear regression to the mean we determined that in these samples three days elapsed between N-protein peak and seroconversion (FIG. 11C day 10 to day 13) and ten days elapsed between N-protein peak and IgG plateau (FIG. 11C day 10 to day 20). We defined seroconversion here as an increase to 5% of the max level measured. To investigate the possibility of post-seroconversion antigen masking influencing measured levels of N-protein, we treated longitudinal samples from patient 4 with DTT to separate potential antigen-antibody complexes. We observed a negligible impact on N-protein levels, suggesting that antigen clearance, rather than antigen masking, causes the observed decrease in N-Protein concentration (FIG. 17).

Dried blood microsamples (DBS). A total of 62 DBS samples were collected from 22 PCR+ and 15 PCR− individuals over multiple weeks in the presence of active COVID-19 infections from CTCH, a long-term care facility that established weekly testing of residents and staff using an FDA-authorized molecular test. An additional 64 PCR− samples were collected from a commercial source. Days of collection relative to initial PCR for the CTCH are shown in Table 5; full data is shown in supplementary information source data file on tab “CTCH Characteristics”.

TABLE 5
Sampling and testing timeline in CTCH study.

| Collection Number | Collection 1 | Collection 2 | Collection 3 |
|---|---|---|---|
| Relative Day of PCR | 1 | 8 | No data |
| Relative Day of DBS | 5 | 12 | 29 |
| Collection | | | |
| Number of PCR+ | 11 | 9 | 14 |
| Donors | | | |
| Number of PCR− | 9 | 13 | 7 |
| Donors | | | |
| Notes | After | After | Follow up |
| | collection 4 | collection 1 | timepoint |
| | donors died, | donor died, | that does not |
| | 1 declined, 7 | 11 declined, | include |
| | new enrolled. | 10 new | recent PCR+ |
| | | enrolled. | infections. |

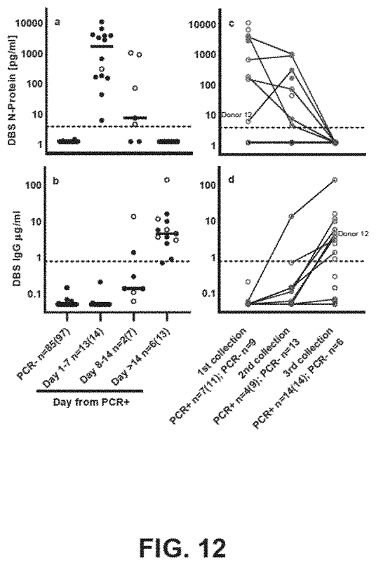

FIG. 12A shows data binned by day from initial PCR test. Using a preliminary cutoff of 3.91 pg/mL, data demonstrate 100% NPA and PPA of the N-protein assay relative to RT-PCR for days 1-7. N-antigen is undetectable after two weeks post-PCR and IgG levels increase concomitantly. FIG. 12B compares N-protein and IgG levels from individual donors over three collections (Table 5). In most donors antigen monotonically decreases while IgG increases. A notable exception is Donor 12, a staff member who had N-Protein levels above cutoff before developing symptoms or obtaining a positive PCR test, whose N-protein levels increased almost 50-fold by time of second collection one week later. Nine donors had levels of N-Protein above cutoff, some over multiple collections for a total 11 samples, despite an absence of SARS-CoV-2 symptoms (open red symbols). Anti-SARS-CoV-2 spike IgG levels increased above cutoff for one PCR+ donor by 2^{nd} collection and for 13 of 14 donors at 3^{rd} collection.

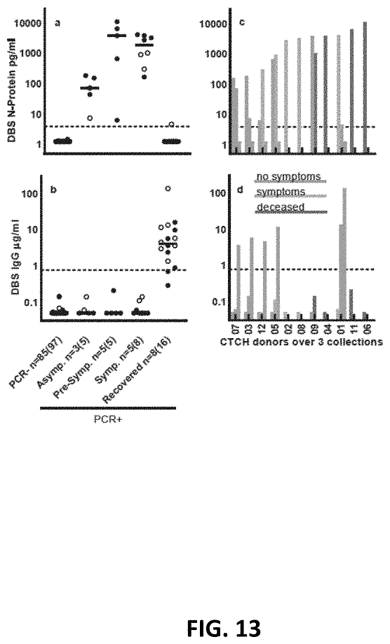

In FIG. 13 we present DBS data from these same donors, sorted by severity of reported symptoms. FIG. 13A shows PCR− donors and PCR+ donors sorted into: asymptomatic (donors with very mild or no symptoms reported throughout the study); pre-symptomatic (donors reported as symptom-free at initial collection but and reported with symptoms at a subsequent timepoint); symptomatic (including donors with symptoms reported at first test and pre-symptomatic donor timepoints after development of symptoms); recovered (including novel donors>14 days after positive PCR test or with IgG above cutoff, pre-symptomatic and symptomatic donors after IgG increased above cutoff). Asymptomatic donors had a median level of N-protein of 72 pg/mL, median levels increased markedly in pre-symptomatic (3896 pg/mL) and symptomatic (1931 pg/mL) donors. Upon recovery from symptoms, N-protein mostly disappeared from the blood (7 or 8 donors below cutoff), decreases in N-protein being accompanied by a corresponding increase in IgG.

We ranked PCR+ donors by increasing N-protein level (from left to right), and color-coded results according to disease severity, defined from best to worst as: no symptoms (includes asymptomatic and pre-symptomatic); symptoms; deceased (FIG. 13B). Worse disease severity associates with higher N-protein level: a two-sided Wilcoxon test showed a statistically significant difference in median N-protein levels between the no symptom (n=5; 186.1 pg/ml) and symptom/deceased groups (n=6; 4079.9 pg/ml) for the first collection (p=0.0173). We observe concomitant IgG increase occurring for most donors at the 3^{rd collection. Donor} 1 was the only donor with N-protein>1000 pg/ml to recover and was the only donor to have anti-spike IgG levels above cutoff by the 2^{nd} collection, suggestive that the early IgG response was protective.

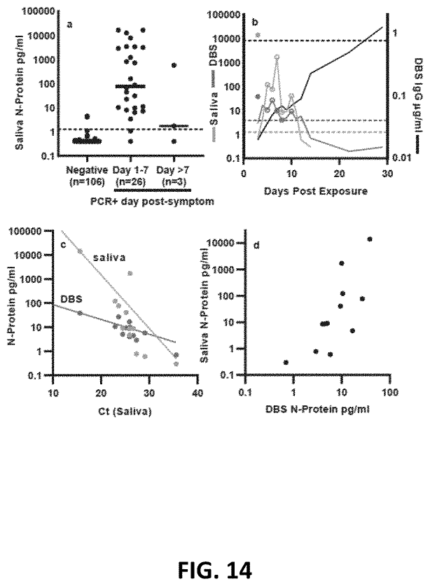

Saliva. FIG. 14A shows the levels of N-protein for 25 pre-pandemic and 81 SARS-CoV-2 PCR− and 29 PCR+ saliva samples binned by days post-symptom. Applying a preliminary cutoff of 1.25 pg/mL to the saliva data demonstrates 98.1% NPA and 92.3% PPA for the N-protein assay for day 1-7.

In a separate case study, we were able to use data from longitudinal, matched saliva and DBS samples to examine the kinetics and relative abundance of N-protein in these matrices over the course of an emerging infection from two donors that co-occupied a shared residence (FIG. 14B). The index case donor developed moderate SARS-CoV-2 symptoms and tested PCR positive by nasal swab on day one. The housemate donor tested PCR negative on day three but developed mild symptoms on day five. Matched Saliva and DBS were positive for N-protein in the index case on day of first antigen test (day 3) and the housemate one day before symptom onset (day 4). Daily sampling of the housemate revealed levels above cutoff until day 11 in saliva and day 12 in DBS. The housemate remained symptomatic from day five until day eight, with symptoms occurring on days with the highest levels of N-protein. Interestingly, symptoms resolved on day nine after N-protein decreased, only to re-occur on day 10 along with a second N-protein peak. Anti-SARS-CoV-2 IgG monitored in DBS increased slowly, perhaps due to the mildness of symptoms, rising above cutoff on day 29.

We quantified RNA levels in the index case and housemate samples using a molecular test with EUA approval for saliva¹⁸. FIG. 14C shows correlation of Ct-values of the N-gene RNA with N-protein in saliva and DBS, with correlation coefficient to the log 2 transform of N-protein of −0.82 and −0.86, respectively. N-protein levels in saliva and DBS also correlate with a coefficient of 0.77 (FIG. 14D). In general, we observe higher and more variable levels of N-protein in saliva than in blood, although the overall distribution of levels is lower than observed in nasopharyngeal swabs (FIG. 20).

Discussion

We describe an ultrasensitive immunoassay which measures SARS-CoV-2 N-Protein in venous blood, dried blood microsamples and saliva. In all matrices we were able to detect N-Protein in >90% of COVID-19 PCR+ positive donors, including those without symptoms. Although these data should not be considered as clinical validation, they strongly suggest that prospective clinical validation studies are merited.

In striking contrast to the high positivity levels of antigen in blood, SARS-CoV-2 RNAemia appears in a much lower percentage of patients than antigen, reported as ranging from 19.6 to 44%, though it correlates with worse disease outcome^{13,15,19,20}. This may be due to RNA being labile in circulation²¹. Ogata et al. also found S1 antigen levels in blood correlate with worse disease outcome, however they detected antigen (S1 or N-protein) in only 48 of 64 patients with severe symptoms²². This may be due to assay differences, because >90% PPA of N-protein measurements in blood was observed for SARS²³ and recently confirmed for SARS-CoV-2²⁴.

Successful detection of SARS-CoV-2 antigen in dried blood microsamples (DBS) suggests potential feasibility of at-home collection. This method requires only 20 μL of capillary blood from a finger-stick, and specimens may be stored and shipped without cold-chain requirements. We report PPA>90% for DBS samples from Day 1-7 post PCR test. We observed lower levels of N-protein in DBS of asymptomatic compared to symptomatic patients; interestingly we also observed a marked increase in pre-symptomatic DBS. This correlates with measurement showing the highest viral load in throat swabs before symptoms, from which peak infectiousness was also inferred to peak before symptom onset²⁵. N-protein levels correlated with worse disease outcome in samples tested here, as has been observed for antigen previously²² and viral RNA as well¹³.

In saliva, also potentially suitable for home collection, we detected N-Protein in >90% of COVID-19 PCR positive donors. When analyzing N-protein in longitudinal saliva and DBS samples from an infected donor, we observed that N-protein presented in both saliva and blood before symptom onset, and that N-protein levels correlate with Ct-values for RNA in saliva, as has been recently observed for N-protein in NP swabs²⁶. Recent work suggests that viral load in saliva is a predictor of mortality²⁷.

In all matrices, N-protein clearance was inversely correlated with an increase in SARS-CoV-2 anti-spike IgG. Seroconversion has been reported to occur between day 7 to 13 post-symptom²⁸, thus based on our longitudinal data in plasma we estimate N-protein peaks 4-10 days post-symptom, similar to timelines previously observed for SARS²³ and SARS-CoV-2²². Our data preliminarily suggests that early IgG response alleviates severe disease outcome, even when high levels of N-protein present.

False negative PCR results have represented a significant challenge during the COVID-19 pandemic, particularly before onset of symtomps^{4,5,29}. Compounding the problem of poor clinical discrimination in pre-symptomatic patients, He et al. observed the highest viral load in throat swabs at time of symptom onset, and inferred that infectiousness will peak at or before symptom onset²⁵. In this context, the high PPA of the Simoa SARS-CoV-2 N-Protein assay across multiple matrices may have utility to detect asymptomatic and pre-symptomatic individuals, although controlled clinical evaluation studies are required.

There are limitations to this work, particularly a limited availability of samples and incomplete clinical annotation for some samples. The U. Bonn and BocaBio cohorts tested (FIG. 11) were predominantly from hospitalized patients, reflecting N-Protein levels from severe infection. Most CTCH samples (FIGS. 12 & 13) were from residents predominantly of older age. We correlate saliva and DBS levels from only two donors (FIG. 14). We report PPA and NPA for the same retrospective samples in which we determined our cutoff, not on a separate or prospective cohort. The cutoffs described herein are preliminary and may change upon further investigation. In a separate but related study, a NIH-RADx supported prospective sample collection is now ongoing, which will enable characterization of this Simoa N-protein test in a larger cohort of prospectively collected samples across multiple matrices. We also note that the HD-X instrument required for Simoa sample analysis is a laboratory-based instrument, therefore this Simoa N-protein test has associated instrument, setup, and consumable costs. Use of automated lab instrumentation does provide throughput benefits; >1000 samples tested per 24-hour period is possible per HD-X analyzer. Furthermore, many of the supply chain shortages that have limited molecular testing³⁰ will not impact a Simoa blood/saliva test, since the Simoa test does not require transport media or swabs, and does not rely on RNA extraction and amplification.

This study demonstrates that the Simoa SARS-CoV-2 N-Protein assay readily detects viral antigen in active, pre-symptomatic and asymptomatic COVID-19 infections in blood and saliva, using sample collection methods that avoid swabs and the need to sample nasopharyngeal or nasal fluids. In addition to utility in studying the kinetics of SARS-CoV-2 infection, this assay may help expand the arsenal of SARS-CoV-2 antigen tests beyond nasal and nasopharyngeal swabs and enable blood- and/or saliva-based detection. Clinical validation studies are ongoing.

Simoa data shown herein was generated using Research Use Only reagents, not In Vitro Diagnostic reagents or devices. The blood and saliva test described in this manuscript has not received an Emergency Use Authorization and is not available in the United States for SARS-CoV-2 diagnostic uses.

Methods

Samples. Healthy pre-pandemic serum and plasma samples (collected before December 2019) were obtained from BiolVT (Westbury, N.Y.). Commercially sourced serum and plasma samples from COVID-19 positive donors, as demonstrated by positive RT-PCR test, were obtained from Boca Biolistics (Pompano Beach, Fla.; hereafter ‘BocaBio’). Samples were collected between Apr. 6 and Jun. 17, 2020. RT-PCR was performed between Mar. 6 and Jun. 12, 2020. Plasma samples from hospitalized COVID-19 patients, as demonstrated by positive RT-PCR test, were provided by Drs. Jacob Nattermann, University of Bonn, Germany. Samples were collected between Mar. 30 and Apr. 22, 2020. RT-PCR was performed between Mar. 30 and Apr. 15, 2020. In COVID-19 patients who were not able to consent at the time of study enrollment, consent was obtained after recovery. Dried blood microsamples (DBS) were collected using Mitra® Devices (Neoteryx, Torrance, Calif.) from staff and residents of Connecticut Baptist Care Homes Inc. (CTCH cohort). COVID-19 status of each donor was determined by RT-PCR test and DBS samples were collected at two time points, one week apart, for measurement of N-protein and IgG levels by Simoa. All staff and residents provided written informed consent prior to participating. Commercial saliva samples (pre-pandemic, PCR negative and PCR positive) were sourced from Lee Biosolutions (Maryland Heights, Mo.). Matched DBS and saliva samples were (PCR positive) were collected from consented donors within Quanterix. Additional PCR negative DBS and saliva samples were collected by Pharos Health (Baton Rouge, La.) from consented donors. The Univ. of Bonn study was approved by the Institutional Review board of the University Hospital Bonn (134/20). All participants in other studies signed written informed consent prior to enrollment; samples were collected under an IRB exemption since these were fully de-identified samples.

Inactivated Virus. Gamma-inactivated SARS-CoV-2 virus was obtained from BEI (beiresources.org), heat-inactivated SARS-CoV-2 and microbial specimens for cross-reactivity testing were obtained from ZeptoMetrix. (zeptometrix.com).

Clinical characteristics. Symptoms from the CTCH cohort were reported by the facility director. Symptoms from the matched DBS-saliva donors (co-residents) were as self-reported. In all cases symptoms were reported before data collection. Days from positive PCR are used in FIG. 11, FIG. 12 and FIG. 13. Days from symptom onset were used in FIG. 14A and days from exposure used in FIG. 14B.

Positive/Negative Cutoff. For N-protein cutoff an ROC analysis of initial sample from single donors was performed for serum/plasma and saliva. For N-protein in DBS multiple timepoints per donor were used due to a limited number of positive samples, and ROC analysis was confined to samples collected within 14 days of PCR. Any concentrations measured below the limit of detection (LOD) for each assay were replaced with the LoD (see Assay Development in Methods). The Youden Index cutoff was compared to the functional limit of quantitation (fLOQ), and the positive/negative cutoff was chosen as the higher of the two. Cutoffs below fLoQ were avoided due to high variance which may impact ability to assess positive and negative samples. Table 6 shows cutoff determination samples and statistics. ROC curves are shown in FIG. 15 and statistics in the source data tabs SI FIG. 11A, SI FIG. 11B, and SI FIG. 11C. Multiple timepoints from individual donors were used for DBS ROC analysis, due to a limited number of positive samples.

TABLE 6
Cutoff determination for three matrices.

| | Serum/Plasma | DBS | Saliva |
|---|---|---|---|
| negative n | 100 | 97 | 106 |
| positive n | 40 | 21 | 29 |
| Youden index | 0.89 | 3.05 | 0.88 |
| fLOQ | 1.25 | 3.91 | 1.25 |
| cutoff chosen | 1.25 | 3.91 | 1.25 |

The cutoffs for the N-protein assay are considered preliminary and may change upon further investigation.

The positive/negative cutoff for the IgG assay was determined during development of the Simoa SARS-CoV-2 Spike IgG assay, and more information can be found in the Instructions for Use of the EUA https://www.fda.gov/media/144764/download.

Software and Statistics. Data were collected using the Simoa HD-X analyzer using Simoa HD-X software, version 3.0.2003.04001. Statistical analyses were performed using Graphpad prism (version 8.4.0 (671), Microsoft Excel (16.0.13530.20132) or R studio, R v.4.0.3 (package pROC)³¹. Non-linear regression to the mean (FIGS. 11B and 11C) were done using either a Lorentzian (N-protein in FIG. 11C) or a 4PL (N-Protein in FIG. 11B, IgG in FIGS. 11B and 11C) logistic equations. To differentiate CTCH “no symptom” from “symptom groups” (collection 1) a two-sided Wilcoxon test was used to determine whether the median N protein level on Day 5 in asymptomatic patients was different from the median N protein in symptomatic patients. The median measured N protein levels on Day 5 in symptomatic patients (4079.85) was higher than the median measured N protein levels in asymptomatic patients (186.10) and the Wilcoxon test showed that the difference was statistically significant (p=0.0173). Correlations were calculated in Excel.

Assay Development. Single Molecule Array (Simoa) technology offers analytical sensitivity on average 1000-fold greater than traditional immunoassay^{32,33}. In brief, the technology involves performing a paramagnetic microbead-based sandwich ELISA, followed by isolation of individual capture beads in arrays of femtoliter-sized reaction wells. Singulation of capture beads within microwells permits buildup of fluorescent product from an enzyme label, so that signal from a single immunocomplex can be detected with a CCD camera in 30 seconds. At very low analyte concentrations, Poisson statistics dictate that bead-containing microwells in the array will contain either a single labeled analyte molecule or no analyte molecules, resulting in a digital signal of either “active” or “inactive” wells. Data collection involves counting active wells corresponding to single enzyme labels. At higher analyte concentrations, digital measurements transition to analog measurements of total fluorescence intensity. Simoa data are reported as Average Enzymes per Bead (AEB). It is widely used in the field of neurodegenerative disease and recently, for the measurement of SARS-CoV-2-associated biomarkers^{34,35}. It has also been demonstrated to rival the sensitivity of PCR for monitoring HIV infection through measurement of the p24 capsid protein in blood^{36,37}.

SARS-CoV-2 N-protein Assay. Antibodies and antigens were obtained from commercial sources. Eight different antibodies and five antigens were screened, resulting in more than 60 different test configurations. The antibody and antigen combination that produced the best signal/background ratio for both calibrator and positive samples was selected. Diluent formulations, detector antibody and Streptavidin-β-Galactosidase concentrations were then optimized, as well as assay protocols (2-step vs 3-step; incubation times). A phosphate-based sample diluent was selected with EDTA to inhibit proteases, heterophilic blocker and a detergent to help de-envelope and inactivate virus particles. For more information on assay performance and validation, including analytical limit of detection (LoD) and limit of quantification (LoQ), see https://www.quanterix.com/simoa-assay-kits/sars-cov-2-n-protein-antigen/. The Simoa® SARS CoV-2 N Protein Advantage Kit is commercially available through Quanterix Item #103806.

SARS-CoV-2 IgG Assay. An assay was developed to monitor the serological response of IgG to the full-spike of SARS-CoV-2. Details of the research use version of this assay can be found at https://www.quanterix.com/simoa-assay-kits/sars-cov-2-spike-igg/. The USFDA recently authorized the Simoa Semi-Quantitative SARS-CoV-2 IgG Antibody Test for Emergency Use—further details are available at https://www.fda.gov/media/144764/download. The Simoa® SARS-CoV-2 Spike IgG Advantage Kit is commercially available through Quanterix Item #103769.

Sample Types. Serum and plasma were collected by normal processing methods and stored frozen at −80° C. before analysis. Serum and plasma samples were diluted 4-fold into assay diluent on the HD-X instrument before measurement. Dried blood spots (DBS) were collected using Mitra collection kits from Neoteryx according to standard protocols (https://www.neoteryx.com/home-blood-blood-collection-kits-dried-capillary-blood). Tips absorb 20 μL of whole blood and are then allowed to dry for at least 16 hours in a pouch with desiccant. Tips are extracted into 250 μL of assay diluent with shaking at 400 rpm overnight at 2-8° C., resulting in a 12.5-fold sample dilution. No further on-board dilution is applied. Saliva samples were collected in polypropylene tubes without preservative and were stored frozen at −80° C. until day of test. Saliva was clarified by centrifuging at 3000×g for 10 minutes before testing, and diluted 4-fold on the HD-X. All sample results have been corrected for dilution factors, to represent the concentration within the sample matrix.

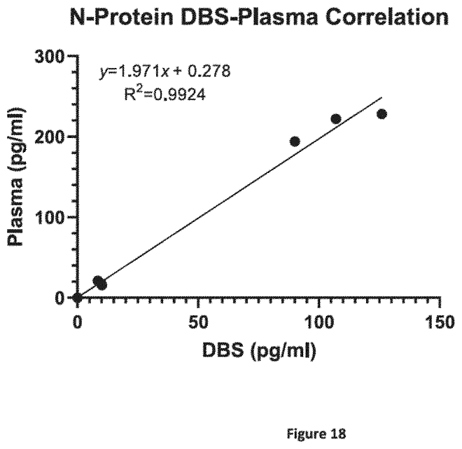

Sample Matrix Correlation. To correlate serum and plasma matrices, matched samples from PCR+ donors were measured with the N-Protein assay. N-protein levels correlated between matrices with a slope of 1.12 and an R² of 0.995 (FIG. 17). To verify the recovery of N-protein from the Mitra tips, whole blood was collected into K2EDTA tubes, spiked with recombinant N-protein, and then processed into either plasma or DBS. N-protein levels were measured in both sample types. N-protein levels correlate between matrices with R²=0.993 and a slope of 1.97. The concentration in DBS was approximately ½ of that in plasma, as expected due to the excluded volume of hematocrit which is separated from plasma (FIG. 18).

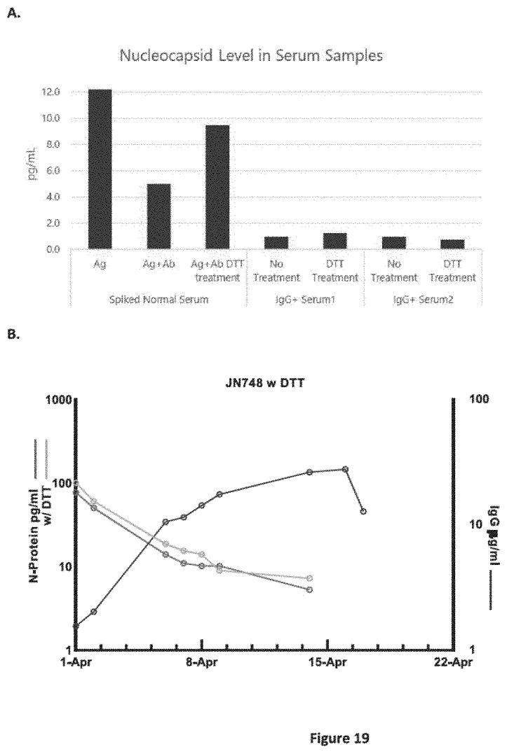

DTT treatment of plasma samples. To determine whether seroconversion and antigen-masking by immunoglobulins plays a role in the decrease of N-protein signal, samples were treated with 10 mM DTT at 37° C. for 15 minutes. To demonstrate the effectiveness of this treatment the following experiment was conducted: 1) negative serum was spiked with N-protein and measured on the N-protein assay; 2) a 500× concentration of anti-N-protein antibody was added and the sample was measured, resulting in a 60% decrease in antigen; 3) the sample spiked with both antigen and antibody was treated with DTT according to the protocol above and measured, resulting in a 75% rescue of antigen signal (FIG. 19).

Cross reactivity studies. Cultured and inactivated pathogens were spiked into negative serum samples to attain 10⁵ TCID50 per ml, using a minimum of 4× dilution of viral stock into serum. Some virus cultures had insufficiently high stock titer to achieve 10⁵ TCID50/mL, and these viruses were tested at the highest titer possible after a 4× dilution into serum. No cross-reactivity was observed, as detailed in Table 7.

TABLE 7
Inactivated, cultured virus was purchased from Zeptometrix, and tested for cross-
reactivity at the TCID₅₀ levels listed. No cross-reactivity was observed.

| | | | Titer | Conc Measured by N | |
|---|---|---|---|---|---|
| | | | Tested | antigen Assay | |
| Virus Description | Vendor | Cat# | TCID₅₀/mL | Serum | Plasma |
| Adenovirus Type 07 | Zeptometrix | 0810021CFHI | 3.52E+04 | <LoD | <LoD |
| (Species B) Culture | | | | | |
| Fluid | | | | | |
| Enterovirus Type 68 | Zeptometrix | 0810237CFHI | 3.78E+05 | <LoD | <LoD |
| (2007 Isolate) Culture | | | | | |
| Fluid | | | | | |
| Influenza A H1N1 (New | Zeptometrix | 0810036CFHI | 2.88E+05 | <LoD | <LoD |
| Ca1/20/99) Culture Fluid | | | | | |
| Influenza B | Zeptometrix | 0810037CFHI | 3.52E+04 | <LoD | <LoD |
| (Florida/02/06) Culture | | | | | |
| Fluid | | | | | |
| Parainfluenza Virus | Zeptometrix | 0810014CFHI | 2.28E+06 | <LoD | <LoD |
| Type 1 Culture Fluid | | | | | |
| Parainfluenza Virus | Zeptometrix | 0810015CFHI | 2.88E+05 | <LoD | <LoD |
| Type 2 Culture Fluid | | | | | |
| Parainfluenza Virus | Zeptometrix | 0810016CFHI | 1.65E+06 | <LoD | <LoD |
| Type 3 Culture Fluid | | | | | |
| Parainfluenza Virus | Zeptometrix | 0810060CFHI | 7.05E+05 | <LoD | <LoD |
| Type 4A Culture Fluid | | | | | |
| Respiratory Syncytial | Zeptometrix | 0810040ACFHI | 9.50E+05 | <LoD | <LoD |
| Virus Type A (Isolate: | | | | | |
| 2006 Isolate) Culture | | | | | |
| Fluid | | | | | |
| Rhinovirus Type 1A | Zeptometrix | 0810012CFNHI | 8.88E+04 | <LoD | <LoD |
| Culture Fluid | | | | | |
| Coronavirus (Strain: | Zeptometrix | 0810229CFHI | 1.04E+05 | <LoD | <LoD |
| 229E) Culture Fluid | | | | | |
| Coronavirus (Strain: | Zeptometrix | 0810024CFHI | 2.63E+05 | <LoD | <LoD |
| 0C43) Culture Fluid | | | | | |
| Coronavirus (Strain: | Zeptometrix | 0810228CFHI | 4.25E+04 | <LoD | <LoD |
| NL63) Culture Fluid | | | | | |

Data Availability. The data that support the findings of this study are available within the manuscript and the supporting information.

### REFERENCES

Cited references are listed below.
1. Max Roser Hannah Ritchie, E. O.-O. & Hasell, J. Coronavirus Pandemic (COVID-19). Our World Data (2020).
2. Amanat, F. et al. A serological assay to detect SARS-CoV-2 seroconversion in humans. Nat. Med. 26, 1033-1036 (2020).
3. Norman, M. et al. Ultrasensitive high-resolution profiling of early seroconversion in patients with COVID-19. Nat. Biomed. Eng. 4, 1180-1187 (2020).
4. Woloshin, S., Patel, N. & Kesselheim, A. S. False Negative Tests for SARS-CoV-2 Infection—Challenges and Implications. N. Engl. J. Med. 383, e38 (2020).
5. Kucirka, L. M., Lauer, S. A., Laeyendecker, O., Boon, D. & Lessler, J. Variation in False-Negative Rate of Reverse Transcriptase Polymerase Chain Reaction-Based SARS-CoV-2 Tests by Time Since Exposure. Ann. Intern. Med. 173, 262-267 (2020).
6. The COVID-19 testing debacle. Nat. Biotechnol. 38, 653 (2020).
7. U.S. Food & Drug Administration. Individual EUAs for Antigen Diagnostic Tests for SARS-CoV-2. https://www.fda.gov/medical-devices/coronavirus-disease-2019-covid-19-emergency-use-authorizations-medical-devices/vitro-diagnostics-euas#individual-antigen.
8. USFDA. Simoa SARS-CoV-2 N Protein Antigen Test. Letter of Authorization https://www.fda.gov/media/144925/download (2021).
9. Li, L. et al. Analysis of viral load in different specimen types and serum antibody levels of COVID-19 patients. J. Transl. Med. 19, 30 (2021).
10. Wyllie, A. L. et al. Saliva or Nasopharyngeal Swab Specimens for Detection of SARS-CoV-2. N. Engl. J. Med. 383, 1283-1286 (2020).
11. Varga, Z. et al. Endothelial cell infection and endotheliitis in COVID-19. Lancet (London, England) 395, 1417-1418 (2020).
12. Wölfel, R. et al. Virological assessment of hospitalized patients with COVID-2019. Nature 581, 465-469 (2020).
13. Fajnzylber, J. M. et al. SARS-CoV-2 Viral Load is Associated with Increased Disease Severity and Mortality. Nat. Commun. 11, (2020).
14. Di Cristanziano, V. et al. Detection of SARS-CoV-2 viremia before onset of COVID-19 symptoms in an allo-transplanted patient with acute leukemia. Bone marrow transplantation (2020) doi:10.1038/s41409-020-01059-y.
15. Eberhardt, K. A. et al. RNAemia Corresponds to Disease Severity and Antibody Response in Hospitalized COVID-19 Patients. Viruses 12, (2020).
16. Bar-On, Y. M., Flamholz, A., Phillips, R. & Milo, R. SARS-CoV-2 (COVID-19) by the numbers. Elife 9, (2020).
17. Ou, J. et al. Emergence of SARS-CoV-2 spike RBD mutants that enhance viral infectivity through increased human ACE2 receptor binding affinity. bioRxiv 2020.03.15.991844 (2020) doi:10.1101/2020.03.15.991844.
18. Infinity BiologiX LLC. Infinity BiologiX TaqPath SARS-CoV-2 Assay. Molecular IFU https://www.fda.gov/media/137773/download (2020).
19. Hogan, C. A. et al. High Frequency of SARS-CoV-2 RNAemia and Association With Severe Disease. Clin. Infect. Dis. (2020) doi:10.1093/cid/ciaa1054.
20. Kawasuji, H. et al. SARS-CoV-2 RNAemia with higher nasopharyngeal viral load is strongly associated with severity and mortality in patients with COVID-19. medRxiv 2020.12.17.20248388 (2020) doi:10.1101/2020.12.17.20248388.
21. Tsui, N. B. Y., Ng, E. K. O. & Lo, Y. M. D. Stability of endogenous and added RNA in blood specimens, serum, and plasma. Clin. Chem. 48, 1647-1653 (2002).
22. Ogata, A. F. et al. Ultra-sensitive Serial Profiling of SARS-CoV-2 Antigens and Antibodies in Plasma to Understand Disease Progression in COVID-19 Patients with Severe Disease. Clin. Chem. 66, 1562-1572 (2020).
23. Che, X.-Y. et al. Nucleocapsid protein as early diagnostic marker for SARS. Emerg. Infect. Dis. 10, 1947-1949 (2004).
24. Hingrat, Q. L. E. et al. Detection of SARS-CoV-2 N-antigen in blood during acute COVID-19 provides a sensitive new marker and new testing alternatives. Clin. Microbiol. Infect. (2020) doi:10.1016/j.cmi.2020.11.025.
25. He, X. et al. Temporal dynamics in viral shedding and transmissibility of COVID-19. Nat. Med. 26, 672-675 (2020).
26. Pollock, N. R. et al. Correlation of SARS-CoV-2 nucleocapsid antigen and RNA concentrations in nasopharyngeal samples from children and adults using an ultrasensitive and quantitative antigen assay. medRxiv 2020.11.10.20227371 (2020) doi:10.1101/2020.11.10.20227371.
27. Silva, J. et al. Saliva viral load is a dynamic unifying correlate of COVID-19 severity and mortality. medRxiv 2021.01.04.21249236 (2021) doi:10.1101/2021.01.04.21249236.
28. Long, Q.-X. et al. Antibody responses to SARS-CoV-2 in patients with COVID-19. Nat. Med. 26, 845-848 (2020).
29. Lauer, S. A. et al. The Incubation Period of Coronavirus Disease 2019 (COVID-19) From Publicly Reported Confirmed Cases: Estimation and Application. Ann. Intern. Med. 172, 577-582 (2020).
30. Supply Shortages Impacting COVID-19 and Non-COVID Testing. American Society for Microbiology httpsliasm.org/Articles/2020/September/Clinical-Microbiology-Supply-Shortage-Collecti-1 (2020).
31. Robin, X. et al. pROC: an open-source package for R and S+ to analyze and compare ROC curves. BMC Bioinformatics 12, 77 (2011).
32. Rissin, D. M. et al. Single-molecule enzyme-linked immunosorbent assay detects serum proteins at subfemtomolar concentrations. Nat. Biotechnol. 28, 595-599 (2010).
33. Wilson, D. H. et al. The Simoa HD-1 Analyzer: A Novel Fully Automated Digital Immunoassay Analyzer with Single-Molecule Sensitivity and Multiplexing. J. Lab. Autom. 21, 533-547 (2016).
34. Kanberg, N. et al. Neurochemical evidence of astrocytic and neuronal injury commonly found in COVID-19. Neurology 95, e1754-e1759 (2020).
35. Ameres, M. et al. Association of neuronal injury blood marker neurofilament light chain with mild-to-moderate COVID-19. J. Neurol. 267, 3476-3478 (2020).
36. Chang, L. et al. Simple diffusion-constrained immunoassay for p24 protein with the sensitivity of nucleic acid amplification for detecting acute HIV infection. J. Virol. Methods 188, 153-160 (2013).
37. Cabrera, C., Chang, L., Stone, M., Busch, M. & Wilson, D. H. Rapid, Fully Automated Digital Immunoassay for p24 Protein with the Sensitivity of Nucleic Acid Amplification for Detecting Acute HIV Infection. Clin. Chem. 61, 1372-1380 (2015).

1. A method for quantitatively detecting a severe acute respiratory syndrome coronavirus 2 (SARS-CoV-2) antigen in a sample, which method comprises:
a) contacting a sample containing or suspected of containing a SARS-CoV-2 antigen with a capture antibody and a detection antibody, said capture antibody or detection antibody comprising a detectable label, under suitable conditions to allow formation of a sandwich complex comprising said SARS-CoV-2 antigen, if present in said sample, said capture antibody and said detection antibody; and
b) assessing a detectable signal from said sandwich complex to assess the amount or level of said SARS-CoV-2 antigen in said sample,
wherein said sample comprises a blood sample or a saliva sample, e.g., a blood sample or a saliva sample from a subject, and said method is conducted using a single molecule array immunoassay.

2. The method of claim 1, wherein the blood sample is plasma, serum, capillary blood, venous blood, dried blood sample or dried blood spot (DBS) sample.

3. The method of claim 2, wherein the blood sample is dried blood spot (DBS) sample.

4. The method of claim 3, wherein the dried blood spot (DBS) sample is obtained from a subject using a foamish-tipped collection device or a collection device comprising an absorbent probe that collects a defined amount of blood, e.g., capillary blood, from the subject.

5. The method of claim 4, wherein the foamish-tipped collection device or a collection device comprising an absorbent probe is Mitra collection device or collection kit from Neoteryx, or a device for collecting bodily fluid described and/or claimed in U.S. Pat. No. 10,531,821 B2, US patent publication No. US 2017/0071520 A1 or US 2013/0116597 A1 or PCT patent publication No. WO 2013/067520 A1.

6. The method of claim 4, wherein about 20 μl of whole blood or capillary blood is collected from the subject.

7. The method of claim 1, wherein the blood sample is a saliva sample.

8. The method of claim 1, wherein the SARS-CoV-2 antigen is a SARS-CoV-2 polypeptide, or a fragment thereof.

9. The method of claim 8, wherein the SARS-CoV-2 polypeptide comprises S (spike) polypeptide, E (envelope) polypeptide, M (membrane) polypeptide, N (nucleocapsid) polypeptide, or a fragment thereof.

10. The method of claim 9, wherein the SARS-CoV-2 polypeptide comprises N (nucleocapsid) polypeptide, or a fragment thereof.

11. The method of claim 1, wherein the capture antibody and/or the detection antibody is an antibody, or a fragment thereof, that specifically binds to the SARS-CoV-2 antigen.

12. The method of claim 11, wherein the capture antibody and/or the detection antibody is an antibody, or a fragment thereof, that specifically binds to the N (nucleocapsid) polypeptide.

13. The method of claim 11, wherein the capture antibody and/or the detection antibody is an antibody is a polyclonal antibody, a monoclonal antibody, or a fragment thereof.

14. The method of claim 1, wherein the detectable label is a colorimetric, radioactive, enzymatic, luminescent or fluorescent label.

15. The method of claim 1, wherein the subject is a mammal.

16. The method of claim 15, wherein the mammal is a human.

17. The method of claim 15, wherein the mammal is non-human mammal, e.g., a non-human primate such as a monkey, a rabbit, or a rodent.

18. The method of claim 1, wherein conducting the single molecule array immunoassay comprises:
forming the sandwich complex comprising the SARS-CoV-2 antigen, the capture antibody and the detection antibody on microparticles;
applying the microparticles to a microfluidic device comprising an array of femtoliter reaction wells and petitioning a single microparticle comprising the sandwich complex in a femtoliter reaction well; and
assessing a detectable signal or signals from the petitioned microparticles to assess the amount or level of the SARS-CoV-2 antigen in the sample.

19. The method of claim 1, which further comprises contacting a sample with a reducing agent, e.g., DTT, to disassociate a SARS-CoV-2 antigen, e.g., SARS-CoV-2 N (nucleocapsid) polypeptide, from an antibody in a sample.

20. The method of claim 1, which is used to quantitatively detect SARS-CoV-2 N (nucleocapsid) polypeptide in a blood sample, e.g., a plasma, serum, dried blood sample or dried blood spot (DBS) sample.

21. The method of claim 20, which:
1) has a specificity ranging from about 80% to about 100%;
2) has a sensitivity ranging from about 80% to about 100%;
3) has a precision (or CV) ranging from about 0% to about 30%; and/or
4) has a detection cut-off from about 0.2 pg/ml to about 10 pg/ml.

22. The method of claim 1, which is used to quantitatively detect SARS-CoV-2 N (nucleocapsid) polypeptide in a saliva sample.

23. The method of claim 22, which:
1) has a specificity ranging from about 80% to about 100%;
2) has a sensitivity ranging from about 80% to about 100%;
3) has a precision (or CV) ranging from about 0% to about 30%; and/or
4) has a detection cut-off from about 1 pg/ml to about 5 pg/ml.

24. The method of claim 1, which further comprises quantitatively detecting an antibody of the subject to SARS-CoV-2.

25. The method of claim 24, wherein quantitatively detecting an antibody of the subject to SARS-CoV-2 comprises quantitatively detecting a class of IgG antibody of the subject to SARS-CoV-2.

26. The method of claim 1, which is used to aid or facilitate diagnosis, prognosis, risk assessment, stratification and/or treatment monitoring of SANS-CoV-2 infection in a subject, and/or for research and drug/vaccine discovery and/or development for treating or preventing SARS-CoV-2 infection.

27. The method of claim 1, which is used to assess SARS-CoV-2 infection and/or recovery status in a subject, e.g., among the following:
1) no infection;
2) infection, asymptomatic;
3) infection, pre-symptomatic;
4) infection, symptomatic; or
5) infection, recovered.

28. A kit or a system for quantitatively detecting a severe acute respiratory syndrome coronavirus 2 (SARS-CoV-2) antigen in a sample, which kit or a system comprises:
a) a capture antibody and a detection antibody, said capture antibody or detection antibody comprising a detectable label, and configured for forming a sandwich complex comprising a SARS-CoV-2 antigen, if present in a sample, said capture antibody and said detection antibody;
b) a device or a kit for collecting a blood sample or a saliva sample from a subject; and
c) a device, a kit, or a reagent for conducting a single molecule array immunoassay.

29. The kit or system of claim 28, wherein the device or a kit for collecting a blood sample comprises a foamish-tipped collection device or a collection device comprising an absorbent probe that collects a defined amount of blood, e.g., capillary blood, from the subject.

30. The kit or system of claim 29, wherein the foamish-tipped collection device or a collection device comprising an absorbent probe is Mitra collection device or collection kit from Neoteryx, or a device for collecting bodily fluid described and/or claimed in U.S. Pat. No. 10,531,821 B2, US patent publication No. US 2017/0071520 A1 or US 2013/0116597 A1 or PCT patent publication No. WO 2013/067520 A1.

31. The kit or system of claim 28, wherein the device or a reagent for conducting a single molecule array immunoassay comprises a single molecule array Simoa immunoassay device, or a reagent to be used in the Simoa immunoassay device.
